(19) United States
(12) Patent Application Publication
(10) Pub. No.: US 2020/0352977 A1
(43) Pub. Date: Nov. 12, 2020

(54) ANTISENSE OLIGONUCLEOTIDES FOR THE TREATMENT OF LEBER CONGENITAL AMAUROSIS

(71) Applicant: STICHTING KATHOLIEKE UNIVERSITEIT, Nijmegen (NL)

(72) Inventors: Robert Wilhelmus Johanna Collin, Venlo (NL); Franciscus Peter Maria Cremers, Malden (NL); Antonia Ingrid Den Hollander, Groesbeek (NL)

(21) Appl. No.: 16/883,723

(22) Filed: May 26, 2020

Related U.S. Application Data
(63) Continuation of application No. 15/508,785, filed on Mar. 3, 2017.
Application No. 15/508,785 is a continuation-in-part of application No. 14/479,229, filed on Sep. 5, 2014, now abandoned.

(51) Int. Cl. A61K 31/712; C12N 15/113; A61P 27/02; C12N 15/85; A61K 31/7125

(52) U.S. Cl. CPC A61K 31/712; C12N 15/113; A61P 27/02; C12N 15/85; A61K 31/7125; C12N 2750/14143; C12N 2330/51; C12N 2310/346; C12N 2320/33; C12N 2310/11; C12N 2310/315; C12N 15/1135

(57) ABSTRACT

The present invention relates to antisense oligonucleotides that are able to induce the skipping of an aberrant 128 nucleotide exon from human CEP290 pre-mRNA and vectors expressing such oligonucleotide.

### CROSS-REFERENCE TO RELATED APPLICATIONS

This application is a continuation of U.S. patent application Ser. No. 15/508,785, filed on Mar. 3, 2017, which is the National Phase of International Patent Application No. PCT/EP2015/070172, filed Sep. 3, 2015, published on Mar. 10, 2016 as WO 2016/034680 A1, which claims priority to U.S. patent application Ser. No. 14/479,229, filed Sep. 5, 2014. The entire contents of each of these applications are herein incorporated by reference.

### SEQUENCE LISTING

The instant application contains a Sequence Listing which has been submitted in ASCII format via EFS-WEB and is hereby incorporated by reference in its entirety. Said ASCII copy, created on May 26, 2020 is named SequenceListing.txt and is 239 KB.

### FIELD OF THE INVENTION

The present invention relates to the fields of medicine and immunology. In particular, it relates to novel antisense oligonucleotides that may be used in the treatment, prevention and/or delay of Leber congenital amaurosis.

### BACKGROUND OF THE INVENTION

Leber congenital amaurosis (LCA) is the most severe form of inherited retinal dystrophy, with an onset of disease symptoms in the first years of life (Leber, T., 1869) and an estimated prevalence of approximately 1 in 50,000 worldwide (Koenekoop et al, 2007; Stone, 2007). Genetically, LCA is a heterogeneous disease, with fifteen genes identified to date in which mutations are causative for LCA (den Hollander et al, 2008; Estrada-Cuzcano et al, 2011). The most frequently mutated LCA gene is CEP290, accounting for ˜15% of all cases (Stone, 2007; den Hollander, 2008; den Hollander, 2006; Perrault et al, 2007). Severe mutations in CEP290 have been reported to cause a spectrum of systemic diseases that, besides retinal dystrophy, are characterized by brain defects, kidney malformations, polydactyly and/or obesity (Baal et al, 2007; den Hollander et al, 2008; Helou et al, 2007; Valente et al, 2006). There is no clear-cut genotype-phenotype correlation between the combination of CEP290 mutations and the associated phenotypes, but patients with LCA and early-onset retinal dystrophy very often carry hypomorphic alleles (Stone, 2007; den Hollander et al, 2006; Perrault et al, 2007; Coppieters et al, 2010; Liitink et al 2010). The by far most frequently occurring hypomorphic CEP290 mutation, especially in European countries and in the US, is a change in intron 26 of CEP290 (c.2991+1655A>G) (Stone, 2007; den Hollander et al, 2006; Perrault et al, 2007; Liitink et al, 2010). This mutation creates a cryptic splice donor site in intron 26 which results in the inclusion of an aberrant exon of 128 bp in the mutant CEP290 mRNA, and inserts a premature stop codon (p.C998X) (see FIGS. 1A and 1B). Besides the mutant CEP290 mRNA, also the wild-type transcript that lacks the aberrant exon is still produced, explaining the hypomorphic nature of this mutation (Estrada-Cuzcano et al, 2011).

LCA, and other retinal dystrophies, for long have been considered incurable diseases. However, the first phase I/II clinical trials using gene augmentation therapy have lead to promising results in a selected group of adult LCA/RP patients with mutations in the RPE65 gene (Bainbridge et al, 2008; Cideciyan et al, 2008; Hauswirth et al, 2008; Maguire et al, 2008). Unilateral subretinal injections of adeno-associated viruses particles carrying constructs encoding the wild-type RPE65 cDNA were shown to be safe and moderately effective in some patients, without causing any adverse effects. In a follow-up study using adults and children, visual improvements were more sustained, especially in the children who all gained ambulatory vision (Maguire et al, 2009). Together, these studies have shown the potential to treat LCA, and thereby enormously boosted the development of therapeutic strategies for other genetic subtypes of retinal dystrophies (den Hollander et al, 2010). However, due to the tremendous variety in gene size, and technical limitations of the vehicles that are used to deliver therapeutic constructs, gene augmentation therapy may not be applicable to all genes. The RPE65 cDNA is for instance only 1.6 kb, whereas the CEP290 cDNA amounts to about 7.4kb, thereby exceeding the cargo size of many available vectors, including the presently used adeno-associated vectors (AAV). In addition, using gene replacement therapy, it is hard to control the expression levels of the therapeutic gene which for some genes need to be tightly regulated. It is therefore an objective of the present invention to provide a convenient therapeutic strategy for the prevention, treatment or delay of Leber congenital amaurosis as caused by an intronic mutation in CEP290.

### DETAILED DESCRIPTION OF THE INVENTION

Surprisingly, it has now been demonstrated that specific antisense oligonucleotides (AONs) are able to block the aberrant splicing of CEP290 that is caused by the intronic LCA mutation.

Accordingly, in a first aspect the present invention provides an exon skipping molecule that binds to and/or is complementary to a polynucleotide with the nucleotide sequence as shown in SEQ ID NO: 6, preferably SEQ ID NO: 17, more preferably SEQ ID NO: 8, even more preferably SEQ ID NO: 7, or a part thereof.

In all embodiments of the present invention, the terms “modulating splicing” and “exon skipping” are synonymous. In respect of CEP290, “modulating splicing” or “exon skipping” are to be construed as the exclusion of the aberrant 128 nucleotide exon (SEQ ID NO: 4) from the CEP290 mRNA (see FIGS. 1A and 1B). The term exon skipping is herein defined as the induction within a cell of a mature mRNA that does not contain a particular exon that would be present in the mature mRNA without exon skipping. Exon skipping is achieved by providing a cell expressing the pre-mRNA of said mature mRNA with a molecule capable of interfering with sequences such as, for example, the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA; such molecules are herein referred to as exon skipping molecules The term pre-mRNA refers to a non-processed or partly processed precursor mRNA that is synthesized from a DNA template in the nucleus of a cell by transcription.

The term “antisense oligonucleotide” is understood to refer to a nucleotide sequence which is substantially complementary to a target nucleotide sequence in a pre-mRNA molecule, hrRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions.

The terms “antisense oligonucleotide” and “oligonucleotide” are used interchangeably herein and are understood to refer to an oligonucleotide comprising an antisense sequence.

In an embodiment, an exon skipping molecule as defined herein can be a compound molecule that binds and/or is complementary to the specified sequence, or a protein such as an RNA-binding protein or a non-natural zinc-finger protein that has been modified to be able to bind to the indicated nucleotide sequence on a RNA molecule. Methods for screening compound molecules that bind specific nucleotide sequences are, for example, disclosed in PCT/NL01/00697 and U.S. Pat. No. 6,875,736, which are herein incorporated by reference. Methods for designing RNA-binding Zinc-finger proteins that bind specific nucleotide sequences are disclosed by Friesen and Darby, Nature Structural Biology 5: 543-546 (1998) which is herein incorporated by reference. Binding to one of the specified SEQ ID NO: 6, 7, 8 or 17 sequence, preferably in the context of the aberrant 128 nucleotide CEP290 exon (SEQ ID NO: 4) may be assessed via techniques known to the skilled person. A preferred technique is gel mobility shift assay as described in EP 1 619 249. In a preferred embodiment, an exon skipping molecule is said to bind to one of the specified sequences as soon as a binding of said molecule to a labeled sequence SEQ ID NO: 6, 7, 8 or 17 is detectable in a gel mobility shift assay.

In all embodiments of the invention, an exon skipping molecule is preferably a nucleic acid molecule, preferably an oligonucleotide. Preferably, an exon skipping molecule according to the invention is a nucleic acid molecule, preferably an oligonucleotide, which is complementary or substantially complementary to a nucleotide sequence as shown in SEQ ID NO: 6, preferably SEQ ID NO: 17, more preferably SEQ ID NO: 8, even more preferably SEQ ID NO: 7, or a part thereof as later defined herein.

The term “substantially complementary” used in the context of the present invention indicates that some mismatches in the antisense sequence are allowed as long as the functionality, i.e. inducing skipping of the aberrant 128 nucleotide CEP290 exon (SEQ ID NO: 4), is still acceptable. Preferably, the complementarity is from 90% to 100%. In general this allows for 1 or 2 mismatch(es) in an oligonucleotide of 20 nucleotides or 1, 2, 3 or 4 mismatches in an oligonucleotide of 40 nucleotides, or 1, 2, 3, 4, 5 or 6 mismatches in an oligonucleotide of 60 nucleotides, etc.

The present invention provides a method for designing an exon skipping molecule, preferably an oligonucleotide able to induce skipping of the aberrant 128 nucleotide CEP290 exon (SEQ ID NO: 4). First, said oligonucleotide is selected to bind to one of SEQ ID NO: 6, 7, 8 or 17, or a part thereof as defined later herein. Subsequently, in a preferred method at least one of the following aspects has to be taken into account for designing, improving said exon skipping molecule any further:
The exon skipping molecule preferably does not contain a CpG or a stretch of CpG,
The exon skipping molecule has acceptable RNA binding kinetics and/or thermodynamic properties.

The presence of a CpG or a stretch of CpG in an oligonucleotide is usually associated with an increased immunogenicity of said oligonucleotide (Dorn and Kippenberger, 2008). This increased immunogenicity is undesired since it may induce damage of the tissue to be treated, i.e. the eye. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an oligonucleotide of the invention by detecting the presence of a neutralizing antibody and/or an antibody recognizing said oligonucleotide using a standard immunoassay known to the skilled person.

An increase in immunogenicity may be assessed by detecting the presence or an increasing amount of a neutralizing antibody or an antibody recognizing said oligonucleotide using a standard immunoassay.

The invention allows designing an oligonucleotide with acceptable RNA binding kinetics and/or thermodynamic properties. The RNA binding kinetics and/or thermodynamic properties are at least in part determined by the melting temperature of an oligonucleotide (Tm; calculated with the oligonucleotide properties calculator (www.unc.edu/˜cail/biotool/oligo/index.html) for single stranded RNA using the basic Tm and the nearest neighbor model), and/or the free energy of the AON-target exon complex (using RNA structure version 4.5). If a Tm is too high, the oligonucleotide is expected to be less specific. An acceptable Tm and free energy depend on the sequence of the oligonucleotide. Therefore, it is difficult to give preferred ranges for each of these parameters. An acceptable Tm may be ranged between 35 and 70° C. and an acceptable free energy may be ranged between 15 and 45 kcal/mol.

The skilled person may therefore first choose an oligonucleotide as a potential therapeutic compound as binding and/or being complementary to SEQ ID NO: 6, 7, 8 or 17, or a part thereof as defined later herein. The skilled person may check that said oligonucleotide is able to bind to said sequences as earlier defined herein. Optionally in a second step, he may use the invention to further optimize said oligonucleotide by checking for the absence of CpG and/or by optimizing its Tm and/or free energy of the AON-target complex. He may try to design an oligonucleotide wherein preferably no CpG and/or wherein a more acceptable Tm and/or free energy are obtained by choosing a distinct sequence of CEP290 (including SEQ ID NO: 6, 7, 8 or 17) to which the oligonucleotide is complementary. Alternatively, if an oligonucleotide complementary to a given stretch within SEQ ID NO: 6, 7, 8 or 17, comprises a CpG, and/or does not have an acceptable Tm and/or free energy, the skilled person may improve any of these parameters by decreasing the length of the oligonucleotide, and/or by choosing a distinct stretch within any of SEQ ID NO: 6, 7, 8 or 17 to which the oligonucleotide is complementary and/or by altering the chemistry of the oligonucleotide.

At any step of the method, an oligonucleotide of the invention is preferably an olignucleotide, which is still able to exhibit an acceptable level of functional activity. A functional activity of said oligonucleotide is preferably to induce the skipping of the aberrant 128 nucleotide CEP290 exon (SEQ ID NO: 4) to a certain extent, to provide an individual with a functional CEP290 protein and/or mRNA and/or at least in part decreasing the production of an aberrant CEP290 protein and/or mRNA. In a preferred embodiment, an oligonucleotide is said to induce skipping of the aberrant 128 nucleotide CEP290 exon (SEQ ID NO: 4), when the aberrant 128 nucleotide CEP290 exon (SEQ ID NO: 4) skipping percentage as measured by real-time quantitative RT-PCR analysis (is at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 95%, or 100%.

Preferably, a nucleic acid molecule according to the invention, preferably an oligonucleotide, which comprises a sequence that is complementary or substantially complementary to a nucleotide sequence as shown in SEQ ID NO: 6, preferably SEQ ID NO: 17, more preferably SEQ ID NO: 8, even more preferably SEQ ID NO: 7, or part thereof of CEP290 is such that the (substantially) complementary part is at least 50% of the length of the oligonucleotide according to the invention, more preferably at least 60%, even more preferably at least 70%, even more preferably at least 80%, even more preferably at least 90% or even more preferably at least 95%, or even more preferably 98% or even more preferably at least 99%, or even more preferably 100%. Preferably, an oligonucleotide according to the invention comprises or consists of a sequence that is complementary to part of SEQ ID NO: 6, 7, 8 or 17. As an example, an oligonucleotide may comprise a sequence that is complementary to part of SEQ ID NO: 6, 7, 8 or 17 and additional flanking sequences. In a more preferred embodiment, the length of said complementary part of said oligonucleotide is of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 67, 68, 69, 70, 75, 80, 85, 90, 95, 100, 110, 115, 120, 125, 130, 135, 140, 141, 142 or 143 nucleotides. Additional flanking sequences may be used to modify the binding of a protein to the oligonucleotide, or to modify a thermodynamic property of the oligonucleotide, more preferably to modify target RNA binding affinity.

It is thus not absolutely required that all the bases in the region of complementarity are capable of pairing with bases in the opposing strand. For instance, when designing the oligonucleotide one may want to incorporate for instance a residue that does not base pair with the base on the complementary strand. Mismatches may, to some extent, be allowed, if under the circumstances in the cell, the stretch of nucleotides is sufficiently capable of hybridizing to the complementary part. In this context, “sufficiently” preferably means that using a gel mobility shift assay as described in example 1 of EP1619249, binding of an oligonucleotide is detectable. Optionally, said oligonucleotide may further be tested by transfection into retina cells of patients. Skipping of a targeted exon may be assessed by RT-PCR (as described in EP1619249). The complementary regions are preferably designed such that, when combined, they are specific for the exon in the pre-mRNA. Such specificity may be created with various lengths of complementary regions as this depends on the actual sequences in other (pre-)mRNA molecules in the system. The risk that the oligonucleotide also will be able to hybridize to one or more other pre-mRNA molecules decreases with increasing size of the oligonucleotide. It is clear that oligonucleotides comprising mismatches in the region of complementarity but that retain the capacity to hybridize and/or bind to the targeted region(s) in the pre-mRNA, can be used in the present invention. However, preferably at least the complementary parts do not comprise such mismatches as these typically have a higher efficiency and a higher specificity, than oligonucleotides having such mismatches in one or more complementary regions. It is thought, that higher hybridization strengths, (i.e. increasing number of interactions with the opposing strand) are favorable in increasing the efficiency of the process of interfering with the splicing machinery of the system. Preferably, the complementarity is from 90% to 100%. In general this allows for 1 or 2 mismatch(es) in an oligonucleotide of 20 nucleotides or 1, 2, 3 or 4 mismatches in an oligonucleotide of 40 nucleotides, or 1, 2, 3, 4, 5 or 6 mismatches in an oligonucleotide of 60 nucleotides, etc.

An exon skipping molecule of the invention is preferably an isolated molecule.

An exon skipping molecule of the invention is preferably a nucleic acid molecule or nucleotide-based molecule, preferably an (antisense) oligonucleotide, which is complementary to a sequence selected from SEQ ID NO: 6, 7, 8 and 17.

A preferred exon skipping molecule, according to the invention is a nucleic acid molecule comprising an antisense oligonucleotide which antisense oligonucleotide has a length from about 8 to about 143 nucleotides, more preferred from about 8 to 60, more preferred from about 10 to 50 nucleotides, more preferred from about 10 to about 40 nucleotides, more preferred from about 12 to about 30 nucleotides, more preferred from about 14 to about 28 nucleotides, nucleotides, most preferred about 20 nucleotides, such as 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 44 nucleotide or 45 nucleotides.

A preferred exon skipping molecule of the invention is an antisense oligonucleotide comprising or consisting of from 8 to 143 nucleotides, more preferred from about 10 to 50 nucleotides, more preferred from about 10 to 40 nucleotides, more preferred from 12 to 30 nucleotides, more preferred from 14 to 20 nucleotides, or preferably comprises or consists of 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 67, 68, 69, 70, 75, 80, 85, 90, 95, 100, 110, 115, 120, 125, 130, 135, 140, 141, 142 or 143 nucleotides.

In all embodiments of the present invention wherein an exon skipping molecule comprises or consists of an antisense oligonucleotide that binds to or is complementary to at least the part of SEQ ID NO: 6 that comprises the c.2991+1655A>G mutation, said exon skipping molecule preferably comprises an “C” nucleotide on the position complementary to the mutated “G” nucleotide in SEQ ID NO: 6.

In certain embodiments, the invention provides an exon skipping molecule comprising or preferably consisting of an antisense oligonucleotide selected from the group consisting of: SEQ ID NO: 10, SEQ ID NO: 11, SEQ ID NO: 12, SEQ ID NO: 20, SEQ ID NO: 21, SEQ ID NO: 22, SEQ ID NO: 23 and SEQ ID NO: 24.

In a more preferred embodiment, the invention provides an exon skipping molecule comprising or preferably consisting of the antisense oligonucleotide SEQ ID NO: 10. It was found that this molecule is very efficient in modulating splicing of the aberrant 128 nucleotide CEP290 exon. This preferred exon skipping molecule of the invention comprising SEQ ID NO: 10 preferably comprises from 16 to 143 nucleotides, more preferred from 16 to 40 nucleotides, more preferred from 16 to 30 nucleotides, more preferred from 16 to 20 nucleotides, or preferably comprises or consists of 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 11. It was found that this molecule is very efficient in modulating splicing of the aberrant 128 nucleotide CEP290 exon. This preferred exon skipping molecule of the invention comprising SEQ ID NO: 11 preferably comprises from 17 to 143 nucleotides, more preferred from 17 to 40 nucleotides, more preferred from 17 to 30 nucleotides, more preferred from 17 to 20 nucleotides, or preferably comprises or consists of 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 12. It was found that this molecule is very efficient in modulating splicing of the aberrant 128 nucleotide CEP290 exon. This preferred exon skipping molecule of the invention comprising SEQ ID NO: 12 preferably comprises from 18 to 143 nucleotides, more preferred from 18 to 40 nucleotides, more preferred from 18 to 30 nucleotides, more preferred from 18 to 20 nucleotides, or preferably comprises or consists of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 20. It was found that this molecule is very efficient in modulating splicing of the aberrant 128 nucleotide CEP290 exon. This preferred exon skipping molecule of the invention comprising SEQ ID NO: 20 preferably comprises from 44 to 143 nucleotides, more preferably from 44 to 60 nucleotides, more preferably from 44 to 50 nucleotides, preferably comprises or consists of 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 21. It was found that this molecule is very efficient in modulating splicing of the aberrant 128 nucleotide CEP290 exon. This preferred exon skipping molecule of the invention comprising SEQ ID NO: 21 preferably comprises from 8 to 143 nucleotides, more preferably from 45 to 60 nucleotides, more preferably from 45 to 50 nucleotides, or preferably comprises or consists of 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 22. It was found that this molecule is very efficient in modulating splicing of the aberrant 128 nucleotide CEP290 exon. This preferred exon skipping molecule of the invention comprising SEQ ID NO: 22 preferably comprises from 21 to 143 nucleotides, more preferably from 21 to 40 nucleotides, more preferably from 21 to 30 nucleotides, more preferably from 21 to 25 nucleotides, or preferably comprises or consists of 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 23. It was found that this molecule is very efficient in modulating splicing of the aberrant 128 nucleotide CEP290 exon. This preferred exon skipping molecule of the invention comprising SEQ ID NO: 23 preferably comprises from 44 to 143 nucleotides, more preferably from 44 to 60 nucleotides, more preferably from 44 to 50 nucleotides, or preferably comprises or consists of 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

In another more preferred embodiment, the invention provides an exon skipping molecule comprising or preferably consisting of the antisense oligonucleotide SEQ ID NO: 24. It was found that this molecule is very efficient in modulating splicing of the aberrant 128 nucleotide CEP290 exon. This preferred exon skipping molecule of the invention comprising SEQ ID NO: 24 preferably comprises from 23 to 143 nucleotides, more preferably from 23 to 40 nucleotides, more preferably from 23 to 30 nucleotides, more preferably from 23 to 25 nucleotides, or preferably comprises or consists of 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 66, 67, 68, 69, 70, 75, 80, 85, 90, 95, 100, 110, 115, 120, 125, 130, 135, 140, 141, 142 or 143 nucleotides.

An exon skipping molecule according to the invention may contain one of more RNA residues (consequently a “t” residue will be a “u” residue as RNA counterpart), or one or more DNA residues, and/or one or more nucleotide analogues or equivalents, as will be further detailed herein below.

It is preferred that an exon skipping molecule of the invention comprises one or more residues that are modified to increase nuclease resistance, and/or to increase the affinity of the antisense oligonucleotide for the target sequence. Therefore, in a preferred embodiment, the antisense nucleotide sequence comprises at least one nucleotide analogue or equivalent, wherein a nucleotide analogue or equivalent is defined as a residue having a modified base, and/or a modified backbone, and/or a non-natural internucleoside linkage, or a combination of these modifications.

In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report demonstrated triplex formation by a morpholino oligonucleotide and, because of the non-ionic backbone, these studies showed that the morpholino oligonucleotide was capable of triplex formation in the absence of magnesium.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

A further preferred backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent of the invention comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; methoxyethoxy; dimethylaminooxyethoxy; and -dimethylaminoethoxyethoxy. The sugar moiety can be a pyranose or derivative thereof, or a deoxypyranose or derivative thereof, preferably ribose or derivative thereof, or deoxyribose or derivative of A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-0,4′-C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target RNA.

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain embodiments, an antisense oligonucleotide of the invention has at least two different types of analogues or equivalents.

A preferred exon skipping molecule according to the invention comprises a 2′-O alkyl phosphorothioate antisense oligonucleotide, such as 2′-O-methyl modified ribose (RNA), 2′-O-ethyl modified ribose, 2′-O-propyl modified ribose, and/or substituted derivatives of these modifications such as halogenated derivatives.

An effective antisense oligonucleotide according to the invention comprises a 2′-O-methyl ribose with a phosphorothioate backbone.

It will also be understood by a skilled person that different antisense oligonucleotides can be combined for efficiently skipping of the aberrant 128 nucleotide exon of CEP290. In a preferred embodiment, a combination of at least two antisense oligonucleotides are used in a method of the invention, such as two different antisense oligonucleotides, three different antisense oligonucleotides, four different antisense oligonucleotides, or five different antisense oligonucleotides.

An antisense oligonucleotide can be linked to a moiety that enhances uptake of the antisense oligonucleotide in cells, preferably retina cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

An exon skipping molecule according to the invention may be indirectly administrated using suitable means known in the art. When the exon skipping molecule is an oligonucleotide, it may for example be provided to an individual or a cell, tissue or organ of said individual in the form of an expression vector wherein the expression vector encodes a transcript comprising said oligonucleotide. The expression vector is preferably introduced into a cell, tissue, organ or individual via a gene delivery vehicle. In a preferred embodiment, there is provided a viral-based expression vector comprising an expression cassette or a transcription cassette that drives expression or transcription of an exon skipping molecule as identified herein. Accordingly, the present invention provides a viral vector expressing an exon skipping molecule according to the invention when placed under conditions conducive to expression of the exon skipping molecule. A cell can be provided with an exon skipping molecule capable of interfering with essential sequences that result in highly efficient skipping of the aberrant 128 nucleotide CEP290 exon by plasmid-derived antisense oligonucleotide expression or viral expression provided by adenovirus- or adeno-associated virus-based vectors. Expression may be driven by a polymerase III promoter, such as a U1, a U6, or a U7 RNA promoter. A preferred delivery vehicle is a viral vector such as an adeno-associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an oligonucleotide as defined herein. Preferred for the current invention are those vectors wherein transcription is driven from PolIII promoters, and/or wherein transcripts are in the form fusions with U1 or U7 transcripts, which yield good results for delivering small transcripts. It is within the skill of the artisan to design suitable transcripts. Preferred are PolIII driven transcripts. Preferably, in the form of a fusion transcript with an U1 or U7 transcript. Such fusions may be generated as described (Gorman L et al, 1998 or Suter D et al, 1999).

The exon skipping molecule according to the invention, preferably an antisense oligonucleotide, may be delivered as such. However, the exon skipping molecule may also be encoded by the viral vector. Typically, this is in the form of an RNA transcript that comprises the sequence of an oligonucleotide according to the invention in a part of the transcript.

One preferred antisense oligonucleotide expression system is an adenovirus associated virus (AAV)-based vector. Single chain and double chain AAV-based vectors have been developed that can be used for prolonged expression of small antisense nucleotide sequences for highly efficient skipping of the aberrant 128 nucleotide CEP290 exon.

A preferred AAV-based vector for instance comprises an expression cassette that is driven by a polymerase III-promoter (Pol III). A preferred Pol III promoter is, for example, a U1, a U6, or a U7 RNA promoter.

The invention therefore also provides a viral-based vector, comprising a Pol III-promoter driven expression cassette for expression of an antisense oligonucleotide of the invention for inducing skipping of aberrant 128 nucleotide CEP290 exon.

An AAV vector according to the present invention is a recombinant AAV vector and refers to an AAV vector comprising part of an AAV genome comprising an encoded exon skipping molecule according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV8, AAV9 and others. Protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

Preferably, a recombinant AAV vector according to the present invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the present invention comprises a capsid protein shell of AAV serotype 2 and the AAV genome or ITRs present in said vector are derived from AAV serotype 5; such vector is referred to as an AAV 2/5 vector.

More preferably, a recombinant AAV vector according to the present invention comprises a capsid protein shell of AAV serotype 2 and the AAV genome or ITRs present in said vector are derived from AAV serotype 8; such vector is referred to as an AAV 2/8 vector.

More preferably, a recombinant AAV vector according to the present invention comprises a capsid protein shell of AAV serotype 2 and the AAV genome or ITRs present in said vector are derived from AAV serotype 9; such vector is referred to as an AAV 2/9 vector.

More preferably, a recombinant AAV vector according to the present invention comprises a capsid protein shell of AAV serotype 2 and the AAV genome or ITRs present in said vector are derived from AAV serotype 2; such vector is referred to as an AAV 2/2 vector.

A nucleic acid molecule encoding an exon skipping molecule according to the present invention represented by a nucleic acid sequence of choice is preferably inserted between the AAV genome or ITR sequences as identified above, for example an expression construct comprising an expression regulatory element operably linked to a coding sequence and a 3′ termination sequence.

“AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. No. 5,139,941, incorporated herein by reference. The AAV helper functions can be supplied on a AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

Preferably, an AAV genome as present in a recombinant AAV vector according to the present invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

Preferably, an AAV vector according to the present invention is constructed and produced according to the methods in Example 2 herein.

A preferred AAV vector according to the present invention is an AAV vector, preferably an AAV2/5, AAV2/8, AAV2/9 or AAV2/2 vector, expressing an exon skipping molecule according to the present invention comprising an antisense oligonucleotide, wherein said antisense oligonucleotide comprises or consists of a sequence selected from the group consisting of: SEQ ID NO: 10, SEQ ID NO: 11, SEQ ID NO: 12, SEQ ID NO: 20, SEQ ID NO: 21, SEQ ID NO: 22, SEQ ID NO: 23, and SEQ ID NO: 24. More preferably, said antisense oligonucleotide comprises or consists of a sequence selected from the group consisting of: SEQ ID NO: 20, SEQ ID NO: 21, SEQ ID NO: 22, SEQ ID NO: 23, and SEQ ID NO: 24. Even more preferably, said antisense oligonucleotide comprises or consists of a sequence selected from the group consisting of: SEQ ID NO: 22, and SEQ ID NO: 23.

A further preferred AAV vector according to the present invention is an AAV vector, preferably an AAV2/5, AAV2/8, AAV2/9 or AAV2/2 vector, expressing an exon skipping molecule according to the present invention comprising an antisense oligonucleotide, wherein said antisense oligonucleotide consists of a sequence selected from the group consisting of: SEQ ID NO: 10, SEQ ID NO: 11, SEQ ID NO: 12, SEQ ID NO: 20, SEQ ID NO: 21, SEQ ID NO: 22, SEQ ID NO: 23, and SEQ ID NO: 24. More preferably, said antisense oligonucleotide consists of a sequence selected from the group consisting of: SEQ ID NO: 20, SEQ ID NO: 21, SEQ ID NO: 22, SEQ ID NO: 23, and SEQ ID NO: 24. Even more preferably, said antisense oligonucleotide consists of a sequence selected from the group consisting of: SEQ ID NO: 22, and SEQ ID NO: 23. A further preferred AAV vector according to the present invention is an AAV vector, preferably an AAV2/5, AAV2/8, AAV2/9 or AAV2/2 vector, expressing an exon skipping molecule according to the present invention selected from the group consisting of: SEQ ID NO: 10, SEQ ID NO: 11, SEQ ID NO: 12, SEQ ID NO: 20, SEQ ID NO: 21, SEQ ID NO: 22, SEQ ID NO: 23, and SEQ ID NO: 24. More preferably, said exon skipping molecule consists of a sequence selected from the group consisting of: SEQ ID NO: 20, SEQ ID NO: 21, SEQ ID NO: 22, SEQ ID NO: 23, and SEQ ID NO: 24. Even more preferably, said exon skipping molecule consists of a sequence selected from the group consisting of: SEQ ID NO: 22, and SEQ ID NO: 23.
A more preferred AAV vector, preferably an AAV2/5, AAV2/8, AAV2/9 or AAV2/2 vector, is a virion corresponding to one of pAAV-AON1, pAAV-AON2, pAAV-AON3, pAAV-AON4, pAAV-AON5, and pAAV-AON6 as depicted in FIG. 4a.
A further more preferred AAV vector, preferably an AAV2/5, AAV2/8, AAV2/9 or AAV2/2 vector, is a virion corresponding to one of pAAV-AON4 and pAAV-AON5, as depicted in FIG. 4a.

Improvements in means for providing an individual or a cell, tissue, organ of said individual with an exon skipping molecule according to the invention, are anticipated considering the progress that has already thus far been achieved. Such future improvements may of course be incorporated to achieve the mentioned effect on restructuring of mRNA using a method of the invention. An exon skipping molecule according to the invention can be delivered as is to an individual, a cell, tissue or organ of said individual. When administering an exon skipping molecule according to the invention, it is preferred that the molecule is dissolved in a solution that is compatible with the delivery method. Retina cells can be provided with a plasmid for antisense oligonucleotide expression by providing the plasmid in an aqueous solution. Alternatively, a plasmid can be provided by transfection using known transfection agentia. For intravenous, subcutaneous, intramuscular, intrathecal and/or intraventricular administration it is preferred that the solution is a physiological salt solution. Particularly preferred in the invention is the use of an excipient or transfection agentia that will aid in delivery of each of the constituents as defined herein to a cell and/or into a cell, preferably a retina cell. Preferred are excipients or transfection agentia capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection agentia comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self assembly into particles that can deliver each constitutent as defined herein to a cell, preferably a retina cell. Such excipients have been shown to efficiently deliver an oligonucleotide such as antisense nucleic acids to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Lipofectin represents an example of a liposomal transfection agent. It consists of two lipid components, a cationic lipid N-[1-(2,3 dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles.

Polycations such like diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an oligonucleotide, across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an exon skipping molecule for use in the current invention to deliver it for the prevention, treatment or delay of a CEP290 related disease or condition. “Prevention, treatment or delay of a CEP290 related disease or condition” is herein preferably defined as preventing, halting, ceasing the progression of, or reversing partial or complete visual impairment or blindness that is caused by a genetic defect in the CEP290 gene.

In addition, an exon skipping molecule according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognising cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, an exon skipping molecule according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon skipping molecule according to the invention and a further adjunct compound as later defined herein.

If required, an exon skipping molecule according to the invention or a vector, preferably a viral vector, expressing an exon skipping molecule according to the invention can be incorporated into a pharmaceutically active mixture by adding a pharmaceutically acceptable carrier.

Accordingly, the invention also provides a composition, preferably a pharmaceutical composition, comprising an exon skipping molecule according to the invention, or a viral vector according to the invention and a pharmaceutically acceptable excipient. Such composition may comprise a single exon skipping molecule according to the invention, but may also comprise multiple, distinct exon skipping molecules according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington, 2000. Each feature of said composition has earlier been defined herein.

If multiple distinct exon skipping molecules according to the invention are used, concentration or dose defined herein may refer to the total concentration or dose of all oligonucleotides used or the concentration or dose of each exon skipping molecule used or added. Therefore in one embodiment, there is provided a composition wherein each or the total amount of exon skipping molecules according to the invention used is dosed in an amount ranged from 0.1 and 20 mg/kg, preferably from 0.5 and 20 mg/kg.

A preferred exon skipping molecule according to the invention, is for the treatment of a CEP290 related disease or condition of an individual. In all embodiments of the present invention, the term “treatment” is understood to include the prevention and/or delay of the CEP290 related disease or condition. An individual, which may be treated using an exon skipping molecule according to the invention may already have been diagnosed as having a CEP290 related disease or condition. Alternatively, an individual which may be treated using an exon skipping molecule according to the invention may not have yet been diagnosed as having a CEP290 related disease or condition but may be an individual having an increased risk of developing a CEP290 related disease or condition in the future given his or her genetic background. A preferred individual is a human being. In a preferred embodiment the CEP290 related disease or condition is Leber congenital amaurosis.

Accordingly, the present invention further provides an exon skipping molecule according to the invention, or a viral vector according to the invention, or a composition according to the invention for use as a medicament, for treating a CEP290 related disease or condition requiring modulating splicing of CEP290 and for use as a medicament for the prevention, treatment or delay of a CEP290 related disease or condition. A preferred CEP290 related disease or condition is Leber congenital amaurosis. Each feature of said use has earlier been defined herein.

The invention further provides the use of an exon skipping molecule according to the invention, or of a viral vector according to the invention, or a composition according to the invention for the treatment of a CEP290 related disease or condition requiring modulating splicing of CEP290. In a preferred embodiment the CEP290 related disease or condition is Leber congenital amaurosis.

The present invention further provides the use of an exon skipping molecule according to the invention, or of a viral vector according to the invention, or a composition according to the invention for the preparation of a medicament, for the preparation of a medicament for treating a CEP290 related disease or condition requiring modulating splicing of CEP290 and for the preparation of a medicament for the prevention, treatment or delay of a CEP290 related disease or condition. A preferred CEP290 related disease or condition is Leber congenital amaurosis. Therefore in a further aspect, there is provided the use of an exon skipping molecule, viral vector or composition as defined herein for the preparation of a medicament, for the preparation of a medicament for treating a condition requiring modulating splicing of CEP290 and for the preparation of a medicament for the prevention, treatment or delay of a CEP290 related disease or condition. A preferred CEP290 related disease or condition is Leber congenital amaurosis. Each feature of said use has earlier been defined herein. An exon skipping molecule according to the invention, or a viral vector according to the invention, or a composition according to the invention in for the preparation of a medicament according to the invention is preferably administered systemically or intraocularly, preferably intravitreally or subretinally.

A treatment in a use or in a method according to the invention is at least one week, at least one month, at least several months, at least one year, at least 2, 3, 4, 5, 6 years or more. Each exon skipping molecule or exon skipping oligonucleotide or equivalent thereof as defined herein for use according to the invention may be suitable for direct administration to a cell, tissue and/or an organ in vivo of individuals already affected or at risk of developing CEP290 related disease or condition, and may be administered directly in vivo, ex vivo or in vitro. The frequency of administration of an oligonucleotide, composition, compound or adjunct compound of the invention may depend on several parameters such as the age of the patient, the mutation of the patient, the number of exon skipping molecules (i.e. dose), the formulation of said molecule. The frequency may be ranged between at least once in two weeks, or three weeks or four weeks or five weeks or a longer time period.

Dose ranges of an exon skipping molecule, preferably an oligonucleotide according to the invention are preferably designed on the basis of rising dose studies in clinical trials (in vivo use) for which rigorous protocol requirements exist. An exon skipping molecule or an oligonucleotide as defined herein may be used at a dose which is ranged from 0.1 and 20 mg/kg, preferably from 0.5 and 20 mg/kg.

In a preferred embodiment, a concentration of an oligonucleotide as defined herein, which is ranged from 0.1 nM and 1 μM is used. Preferably, this range is for in vitro use in a cellular model such as retina cells or retinal tissue. More preferably, the concentration used is ranged from 1 to 400 nM, even more preferably from 10 to 200 nM, even more preferably from 50 to 100 nm. If several oligonucleotides are used, this concentration or dose may refer to the total concentration or dose of oligonucleotides or the concentration or dose of each oligonucleotide added.

In a preferred embodiment, a viral vector, preferably an AAV vector as described earlier herein, as delivery vehicle for a molecule according to the invention, is administered in a dose ranging from 1×10⁹-1×10¹⁷ virus particles per injection, more preferably from 1×10¹⁰-1×10¹⁴, and most preferably 1×10¹⁰-1×10¹² virus particles per injection.

The ranges of concentration or dose of oligonucleotide(s) as given above are preferred concentrations or doses for in vitro or ex vivo uses. The skilled person will understand that depending on the oligonucleotide(s) used, the target cell to be treated, the gene target and its expression levels, the medium used and the transfection and incubation conditions, the concentration or dose of oligonucleotide(s) used may further vary and may need to be optimized any further.

An exon skipping molecule according to the invention, or a viral vector according to the invention, or a composition according to the invention for use according to the invention may be suitable for administration to a cell, tissue and/or an organ in vivo of individuals already affected or at risk of developing a CEP290 related disease or condition, and may be administered in vivo, ex vivo or in vitro. Said exon skipping molecule according to the invention, or a viral vector according to the invention, or a composition according to the invention may be directly or indirectly administrated to a cell, tissue and/or an organ in vivo of an individual already affected by or at risk of developing a CEP290 related disease or condition, and may be administered directly or indirectly in vivo, ex vivo or in vitro. As Leber congenital amaurosis has a pronounced phenotype in retina cells, it is preferred that said cells are retina cells, it is further preferred that said tissue is the retina and/or it is further preferred that said organ comprises or consists of the eye. Accordingly, an exon skipping molecule according to the invention, or a viral vector according to the invention, or a composition according to the invention for use according to the invention is preferably administered systemically or intraocularly, preferably intravitreally or subretinally.

The invention further provides a method for modulating splicing of CEP290 in a cell comprising contacting the cell, preferably a retina cell, with an exon skipping molecule according to the invention, or a viral vector according to the invention, or a composition according to the invention. The features of this aspect are preferably those defined earlier herein. Contacting the cell with an exon skipping molecule according to the invention, or a viral vector according to the invention, or a composition according to the invention may be performed by any method known by the person skilled in the art. Use of the methods for delivery of exon skipping molecules, viral vectors and compositions described herein is included. Contacting may be directly or indirectly and may be in vivo, ex vivo or in vitro.

The invention further provides a method for the treatment of a CEP290 related disease or condition requiring modulating splicing of CEP290 of an individual in need thereof, said method comprising contacting a cell, preferably a retina cell, of said individual with an exon skipping molecule according to the invention, or a viral vector according to the invention, or a composition according to the invention. The features of this aspect are preferably those defined earlier herein. Contacting the cell, preferably a retina cell with an exon skipping molecule according to the invention, or a viral vector according to the invention, or a composition according to the invention may be performed by any method known by the person skilled in the art. Use of the methods for delivery of molecules, viral vectors and compositions described herein is included. Contacting may be directly or indirectly and may be in vivo, ex vivo or in vitro. A preferred CEP290 related disease or condition is Leber congenital amaurosis. Accordingly, an exon skipping molecule according to the invention, or a viral vector according to the invention, or a composition according to the invention in a method of treatment according to the invention is preferably administered systemically or intraocularly, preferably intravitreally or subretinally.

Unless otherwise indicated each embodiment as described herein may be combined with another embodiment as described herein.

As can be observed in the experimental section herein, at the RNA level, addition of various AONs targeting the aberrant CEP290 exon indeed resulted in a conversion of aberrantly spliced CEP290 mRNA to correctly spliced CEP290 mRNA. This conversion will coincide with an increased synthesis of the wild-type CEP290 protein.

In fibroblasts (that can be derived from skin cells), CEP290 is abundantly expressed. Therefore, it is to be expected that addition of AONs to cultured fibroblasts from LCA patients will result in an increased amount of wild-type CEP290 protein that is detectable on Western blot, and as such will demonstrate that AON-based therapy will not only redirect normal splicing of CEP290 mRNA but will also result in restoring CEP290 protein function. This experiment is presently ongoing.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptide obtainable by expression of the gene present in SEQ ID NO: 1 containing the nucleic acid sequence coding for the polypeptide should prevail.

All patent and literature references cited in the present specification are hereby incorporated by reference in their entirety.

### BRIEF DESCRIPTION OF THE FIGURES

FIG. 1 CEP290 splicing and AON function

A) Normal CEP290 mRNA splicing of exons 26 and 27, resulting in wild-type CEP290 protein. (CEP290 gene portion, SEQ ID NO:51)

B) The most frequent LCA-causing mutation is an A-to-G transition (underlined and indicated with an asterisk) in intron 26 of CEP290. This mutation creates a splice donor site, which results in the inclusion of an aberrant exon to ˜50% of the CEP290 mRNA and subsequent premature termination of the CEP290 protein. (aberrant CEP290 gene portion, SEQ ID NO:52)

C) Upon binding of sequence-specific AONs, factors involved in splicing will not recognize the aberrant splice donor site in intron 26, resulting in redirection of normal CEP290 splicing and synthesis of a correct CEP290 protein. (aberrant CEP290 gene portion, SEQ ID NO:52; exemplary AON, SEQ ID NO:53).

FIGS. 2a, 2b, and 2c. AON-based rescue of aberrant CEP290 splicing

A) RT-PCR analysis of CEP290 mRNA isolated from lymphoblastoid cells of one control individuals and two individuals affected with LCA, that were cultured in the absence or presence of a selected AON (AON-3) direct against the aberrant CEP290 exonin a final concentration of 1.0 μM. The upper band represents the aberrant CEP290 splice product, whereas the lower band represents the wild-type CEP290 splice product. M: 100-bp marker. MQ: negative water control.

B) Specificity of AON-based rescue. Similar to A), cells were transfected with AON-3, or a sense oligonucleotide directed to the same target site (SON-3). Left panel: RT-PCR reaction using primers located in exon 26 and exon 27. Right panel: RT-PCR reaction using primers located in exon 26 and exon 31.

C) Dose-dependent rescue of CEP290 mRNA splicing. Similar to A), cells were transfected with different concentrations of the selected AON, ranging from 0.01 to 1.0 μM.

FIGS. 3a and 3b. Sequence specificity in AON-based rescue of aberrant CEP290 splicing

A) Overview of the aberrant CEP290 exon, and the relative positions of the AONs that were selected. The 5′-end of the aberrant exon is part of an Alu repeat.

B) RT-PCR analysis of CEP290 mRNA isolated from lymphoblastoid cells of an LCA patient that were cultured in the absence or presence of different AONs direct against the aberrant CEP290 exon (AON-1 to -5), or one sense oligonucleotide (SON-3). The AONs and SON were transfected in a final concentration of 0.1 μM. The upper band represents the aberrant CEP290 splice product, whereas the lower band represents the wild-type CEP290 splice product. M: 100-bp marker.

FIGS. 4a, 4b, and 4c. Generated constructs and assessment of minigenes

A) Upper panel: graphical representation of the pSMD2 constructs containing the modified U7snRNA gene and inserted AON sequences. Lower panel: exact sequences of the AONs used in the different constructs, aligned with the sequence of the cryptic exon (“-” is used for alignment purposes, it represents neither a gap nor a nucleotide residue. The intronic c.2991+1655A>G mutation is indicated in bold and underlined. As shown in the figure: nkdAON and pAAV-AON1 (SEQ ID NO:25), pAAV-AON2 (SEQ ID NO:54), pAAV-AON3 (SEQ ID NO:55), pAAV-AON4 (SEQ ID NO:28), pAAV-AON5 (SEQ ID NO:56), pAAV-AON6 (SEQ ID NO: 57), and portion of aberrant CEP290 gene (SEQ ID NO:59).

B) Schematic drawing of the LCA minigene construct, containing the genomic DNA sequence of CEP290 from intron 25 to intron 27, including the c.2991+1655A>G mutation in intron 26. This sequence is flanked by exon 3 and 5 of the RHO gene.

C) RT-PCR analysis of CEP290 on HEK293T cells transfected with the WT or LCA minigene, in comparison with that in fibroblast cells from healthy controls or patients with CEP290-associated LCA.

FIG. 5. Splice correction efficacy of AON-containing vectors

HEK293T cells were co-transfected with the LCA minigene and three different concentrations of the six different pAAV-AON constructs (0.5, 0.125 or 0.035 μg of plasmid DNA). RT-PCR analysis from exon 26 to exon 27 of CEP290 revealed the aberrant transcript that contains the cryptic exon X. pAAV-AON4 and pAAV-AON5 were most effective. Naked AON (nkdAON) was used as a positive control. U7snRNA and RHO amplification were used as a transfection control for the pAAV-AON and the minigene constructs, respectively. Actin was used as a loading control.

FIG. 6. Splice correction efficacy of AON-containing AAVs RT-PCR analysis on two different patient cell lines transduced with AAV-NoAON, AAV-AON4 or AAV-AON5, at three different MOIs (100; 1,000 and 10,000). Amplification from exon 26 to exon 27 of CEP290 revealed the presence of the aberrant transcript in the non-treated (NT) as well as the AAV-NoAON-transduced cells, while it was strongly decreased or completely absent in the AAV-AON4 and AAV-AONS-treated cells. Transfection of the naked AON (nkd AON) sderved as a positive control. U7snRNA amplification was used as a measure for the transduction efficacy, and actin as a loading control.

FIGS. 7a and 7b. Assessment of CEP290 protein levels upon transduction of AON-containing AAVs

A) Immunodetection of CEP290 protein levels in treated and non-treated (NT) LCA fibroblast cells in comparison to the control fibroblast cells (C1 and C2). Tubulin detection was used for normalization.

B) Quantification of CEP290 protein levels shown in panel A. Values were normalized against tubulin. C1 was set up as a 100% and all samples were referred to this value. Naked AON (nkdAON) and AAV-AON4 and AAV-AONS significantly increased the CEP290 protein levels. T-test was performed: *p-value<0.05; **p-value<0.01 and ***p-value<0.001.

FIGS. 8a.1, 8a.2, and 8b. Immunocytochemical analysis of cilium integrity Immunocytochemistry in control (C) and patient (LCA) fibroblast cell lines. CEP290 (in black) localizes to the basal body of the cilia (as indicated by the head arrows). The cilium axoneme is stained with acetylated tubulin (in dark grey) whereas the nuclei are stained with DAPI (dotted grey).

B) Quantification of the percentage of ciliated cells and the length of the cilium in treated and untreated LCA cells compared to control (C1 and C2) cells. A minimum of 150 ciliated cells were measured for each condition and a Mann-Whitney test was used for statistic analysis. **p-value<0.01 and ***p-value<0.001.

FIGS. 9a, 9b, 9c, and 9d. In vivo correction of aberrantly spliced CEP290

A) Representative gel electrophoresis of the PCR reactions amplifying exon 26 to 27, exon 26 to cryptic exon X, U7snRNA and actin (to normalize) of one of each replicate. MQ is the negative control of the PCR. U stands for untreated and refers to PBS-injected retinas, while T means treated and shows the effect on the AON or AAV-AON-injected retinas. B) Schematic representation of the decrease of aberrant exon X in each replicate. Bands were semi-quantified with ImageJ and normalized against actin. The Y axis indicated the arbitrary units (a.u.). C) Percentage of decrease of aberrant exon X. PBS-injected eyes were considered as a reference and placed at 100%. D) Fold-increase of U7snRNA detection. PBS-injected retinas were taken as a reference and set at 1.0. In all graphs, *p-value≤0.05 and **p-value≤0.01.

FIGS. 10a and 10b. Assessment of the structure of the retina after treatment Seven micrometer cryosections stained with toluidine blue. A) 50× magnification images covering the complete retina. B) 400× magnification images. RPE: Retinal Pigment Epithelium; PhL: Photoreceptor Layer; ONL: Outer Nuclear Layer; OPL: Outer Plexiform layer; INL: Inner Nuclear Later; IPL: Inner Plexiform Layer and GCL: Ganglion Cell Layer.

FIG. 11. GFAP immunostaining

Immunostaining of seven μm cryosections from mice treated with PBS, naked AON, AAV-NoAON or AAV-AONS. DAPI (darker grey) stains the nuclei while GFAP (black) is an indicator of gliosis and structural stress in the retina. No differences were observed between PBS injected retinas and molecule injected retinas. RPE: Retinal Pigment Epithelium; PhL: Photoreceptor Layer; ONL: Outer Nuclear Layer; OPL: Outer Plexiform layer; INL: Inner Nuclear Later; IPL: Inner Plexiform Layer and GCL: Ganglion Cell Layer.

### SEQUENCES

All sequences herein are depicted from 5′→3′

TABLE 1
Sequences as set forth in the Sequence Listing

| SEQ | | |
|---|---|---|
| ID | | |
| NO: | SEQ type | Description |
| 1 | Genomic DNA | CEP290 |
| 2 | cDNA | CEP290 |
| 3 | PRT | CEP290 protein |
| 4 | DNA | 128 nucleotide aberrant |
| | | CEP 290 exon |
| 5 | PRT | CEP290 aberrant protein |
| 6 | Polynucleotide | 143 nucleotide motif |
| 7 | Polynucleotide | 42 nucleotide motif |
| 8 | Polynucleotide | 24 nucleotide motif |
| 9 | AON-1 | taatcccagcactttaggag |
| 10 | AON-2 | gggccaggtgcggtgg |
| 11 | AON-3 | aactggggccaggtgcg |
| 12 | AON-4 | tacaactggggccaggtg |
| 13 | AON-5 | actcacaattacaactgggg |
| 14 | SON-3 | cgcacctggccccagtt |
| 15 | PCR primer | tgctaagtacagggacatcttgc |
| 16 | PCR primer | agactccacttgttcttttaaggag |
| 17 | Polynucleotide | 69 nucleotide motif |
| 18 | Nkd AON | aactggggccaggtgcg |
| 19 | (AAV) AON1 | aactggggccaggtgcg |
| 20 | (AAV) AON2 | ccaggtgcggtggctcacatcgtaa |
| | | tcccagcactttaggagg |
| 21 | (AAV) AON3 | gatactcacaattacaactgggggt |
| | | aatcccagcactttaggagg |
| 22 | (AAV) AON4 | ccaggtgcggtggctcacatc |
| 23 | (AAV) AON5 | gatactcacaattacaactggggcc |
| | | aggtgcggtggctcacatc |
| 24 | (AAV) AON3R | gatactcacaattacaactgggg |
| 58 | (AAV) AON6 | tacaactggggccaggtgcggtgg |
| 25 | (pAAV) AON1 | cgcacctggccccagtt |
| 26 | (pAAV) AON2 | gcctcctaaagtgctgggattacga |
| | | tgtgagccaccgcacctgg |
| 27 | (pAAV) AON3 | cctcctaaagtgctgggattacccc |
| | | cagagtaattgtgaatatc |
| 28 | (pAAV) AON4 | gatgtgagccaccgcacctgg |
| 29 | (pAAV) AON5 | gatgtgagccaccgcacctggcccc |
| | | agttgtaattgtgaatatc |
| 30 | (pAAV) | ccccagttgtaattgtgaatatc |
| | AON3R | |
| 57 | (pAAV) AON6 | ccaccgcacctggccccagttgta |
| 31 | CEP 290ex26 | RT-PCR forward primer |
| 32 | CEP 290ex27 | RT-PCR reverse primer |
| 33 | U7snRNA | RT-PCR forward primer |
| 34 | U7snRNA | RT-PCR reverse primer |
| 35 | Rhodopsin | RT-PCR forward primer |
| 36 | Rhodopsin | RT-PCR reverse primer |
| 37 | Actin | RT-PCR forward primer |
| 38 | Actin | RT-PCR reverse primer |
| 39 | (pAAV) AON1 | PCR forward primer |
| 40 | (pAAV) AON1 | PCR reverse primer |
| 41 | (pAAV) AON2 | PCR forward primer |
| 42 | (pAAV) AON2 | PCR reverse primer |
| 43 | (pAAV) AON3 | PCR forward primer |
| 44 | (pAAV) AON3 | PCR reverse primer |
| 45 | (pAAV) AON4 | PCR forward primer |
| 46 | (pAAV) AON4 | PCR reverse primer |
| 47 | (pAAV) AON5 | PCR forward primer |
| 48 | (pAAV) AON5 | PCR reverse primer |
| 49 | (pAAV) AON6 | PCR forward primer |
| 50 | (pAAV) AON6 | PCR reverse primer |

The present invention is further described by the following examples which should not be construed as limiting the scope of the invention.

Unless stated otherwise, the practice of the invention will employ standard conventional methods of molecular biology, virology, microbiology or biochemistry. Such techniques are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); Oligonucleotide Synthesis (N. Gait editor); Nucleic Acid Hybridization (Hames and Higgins, eds.).

### EXAMPLES

### Example 1: Naked AON's

### Materials and Methods

### Design Antisense Oligonucleotides

The 128-bp sequence of the aberrant CEP290 exon that is included into the mutant CEP290 mRNA was analyzed for the presence of exonic splice enhancer motifs using the ESE finder 3.0 program (rulai.cshl.edu/cgi-bin/tools/ESE3/esefinder.cgi?process=home). RNA antisense oligonucleotides were purchased from Eurogentec, and designed with a Tₘ of 58° C., and modified with a 2′-O-methyl group at the sugar chain and a phosphothiorate backbone, and dissolved in phosphate buffered saline.

### Cell Culture

Human B-lymphoblasts cells of LCA patients homozygously carrying the intronic mutation in CEP290 were immortalized by transformation with the Eppstein-Barr virus, as described previously. (Wall F E, 1995). Cells were cultured in RPMI1640 medium (Gibco) containing 10% (v/v) fetal calf serum (Sigma), 1% 10 U/μl penicillin and 10 μg/μl streptomycin (Gibco), and 1% GlutaMAX (Gibco), at a density of 0.5×10⁶ cells/ml. Cells were passaged twice a week.

### Transfection of AONs

A day before transfection, 1.0×10⁶ cells were seeded in each well of a 6-wells plate, in a total volume of 2 ml complete medium. Transfection mixtures were prepared by combining 2.5 μl AON in a desired concentration, or distilled water, 5 μl transfection reagent (ExGen in vitro 500, Fermentas) and 92.5 μl 150 mM NaCl, and incubated at room temperature for 10 minutes, before addition to the cells. Six hours after transfection, 8 ml of low-serum medium (complete medium with only 1% fetal calf serum) was added. Forty-eight hours after transfection, cells were collected and washed with 1×PBS, before directly proceeding to RNA isolation.

### RNA Isolation and RT-PCR

Total RNA was isolated from transfected lymphoblastoid cells using the Nucleospin RNA II isolation kit (Machery Nagel), according to manufacturer's protocol. Subsequently, 1 μg of total RNA was used for cDNA synthesis using the iScript cDNA synthesis kit (Bio-Rad). Five percent of the cDNA was used for each PCR reaction. Part of the CEP290 cDNA was amplified under standard PCR conditions supplemented with 5% Q-solution (Qiagen), and using forward primer tgctaagtacagggacatcttgc (SEQ ID NO: 15) and reverse primer agactccacttgttcttttaaggag (SEQ ID NO: 16) that are located in exon 26 and exon 27 of the human CEP290 gene, respectively. PCR products were resolved on a 1.5% agarose gel. Bands presumably representing correctly and aberrantly spliced CEP290 were excised from the gel, purified using Nucleospin Extract II isolation kit and sequenced from both strands with the ABI PRISM Big Dye Terminator Cycle Sequencing V2.0 Ready Reaction kit and the ABI PRISM 3730 DNA analyzer (Applied Biosystems).

### Introduction

Here, we describe the use of AONs to redirect normal splicing of CEP290 in patient-derived lymphoblast cells, and show a sequence-specific and dose-dependent decrease in levels of aberrantly spliced CEP290, thereby revealing the potential of AON-based therapy to treat CEP290-associated LCA.

### Results

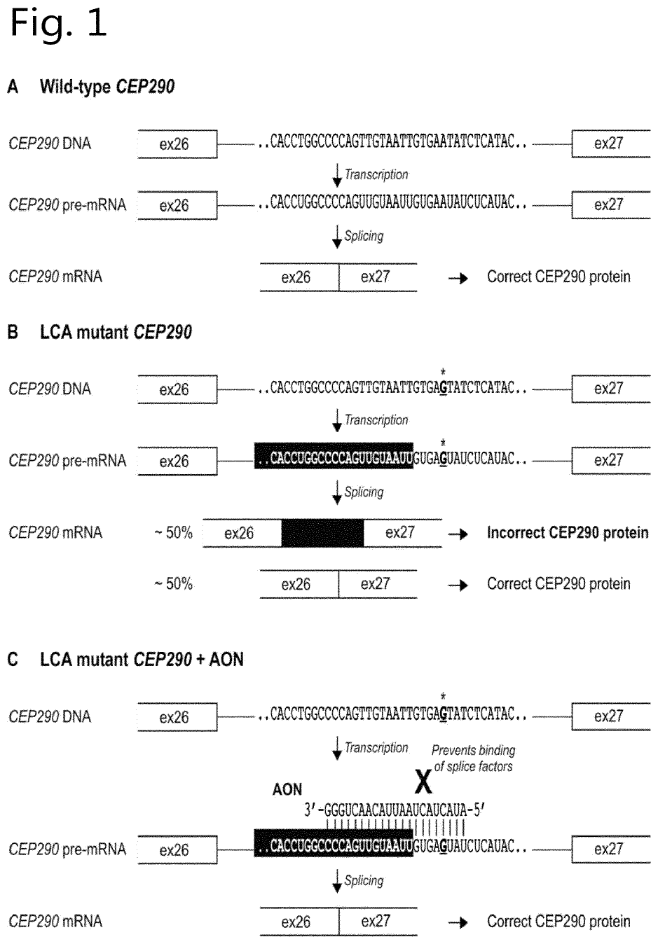

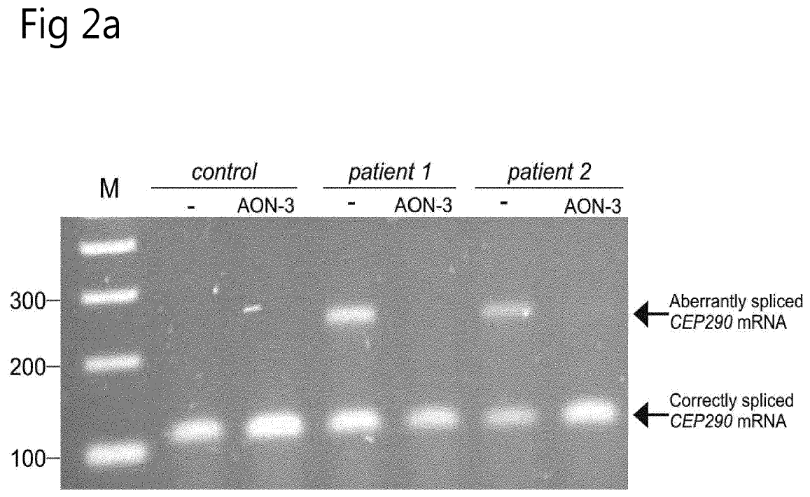

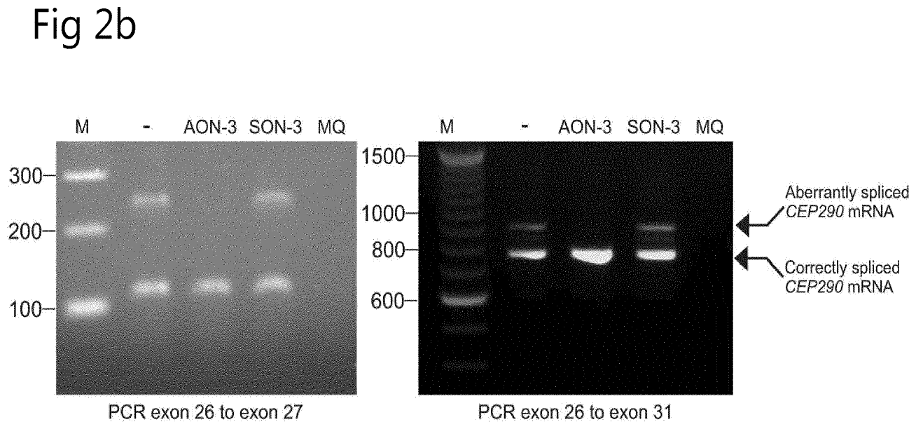

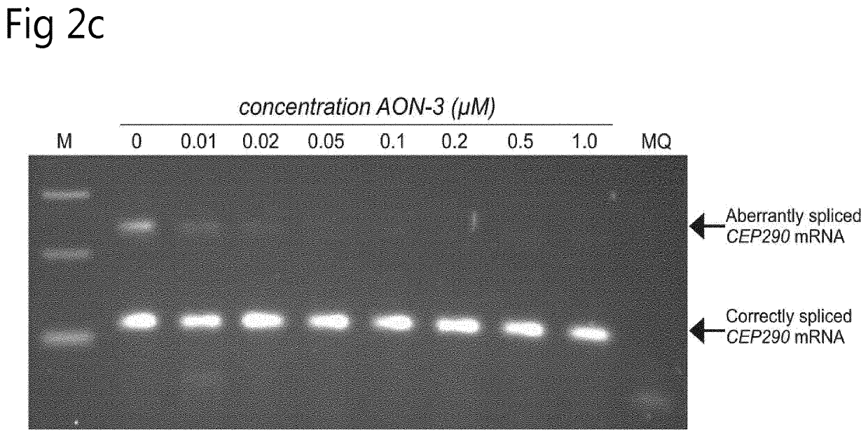

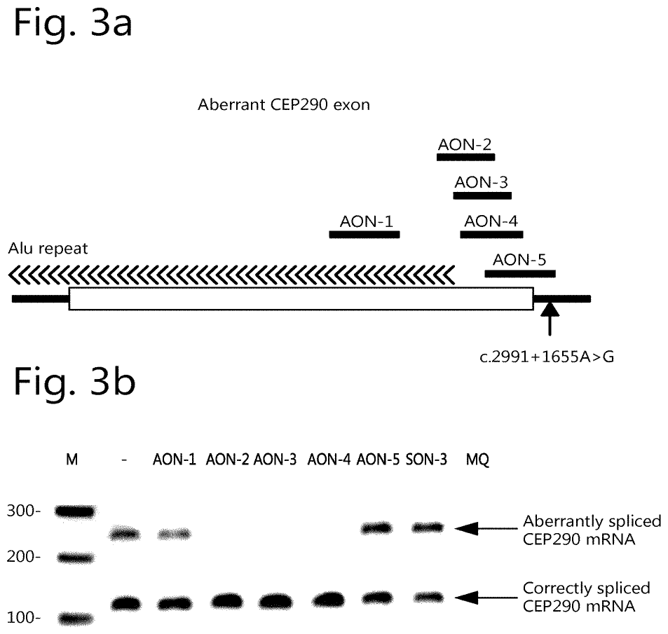

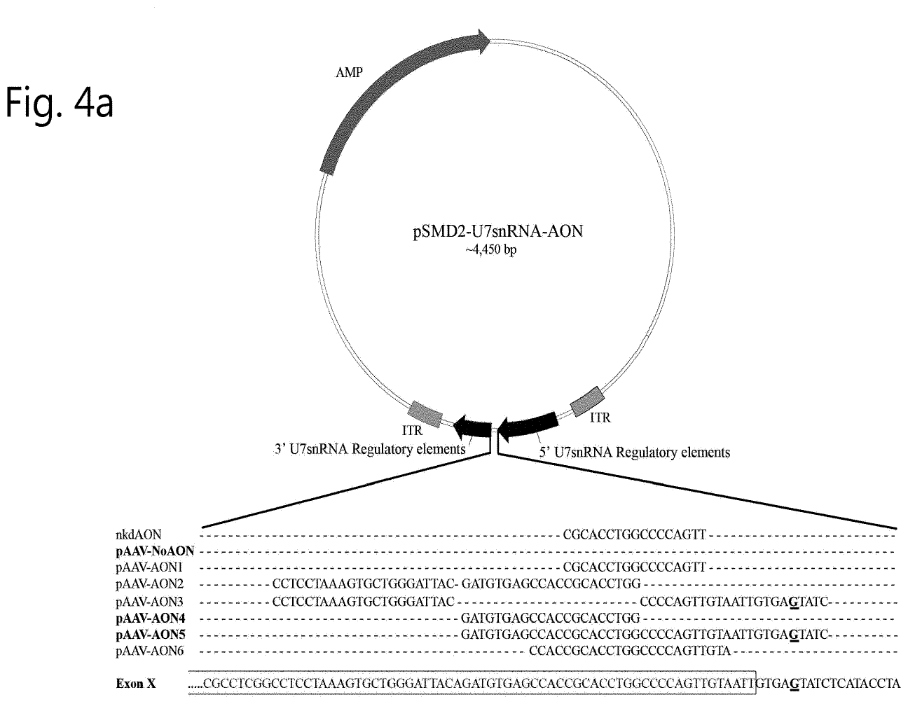

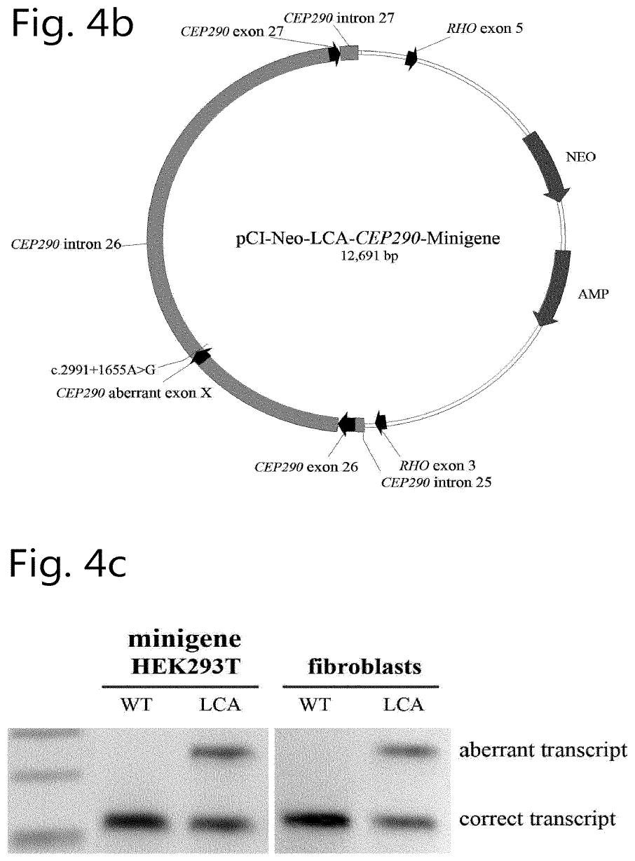

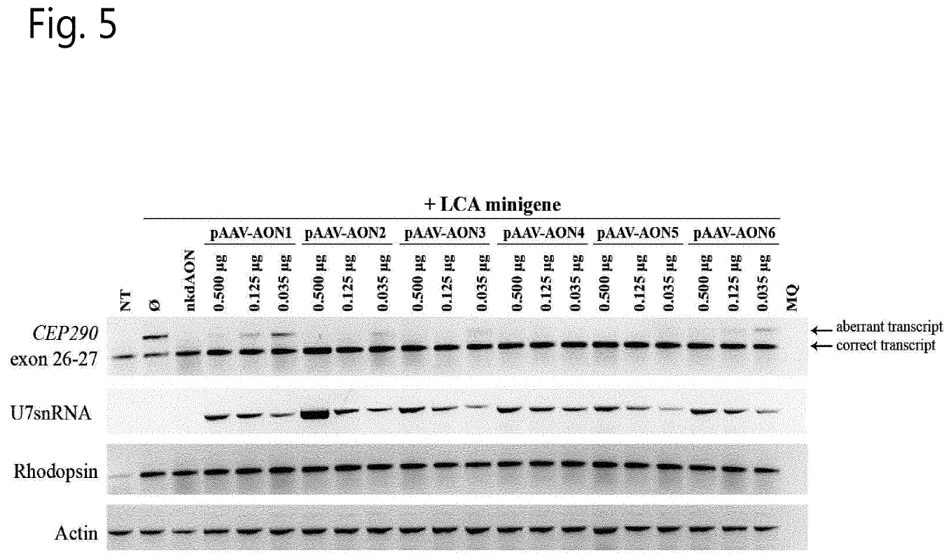

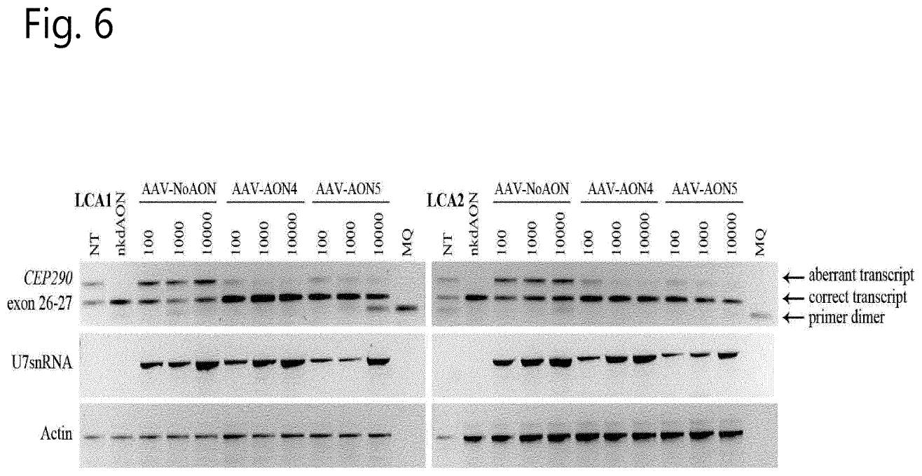

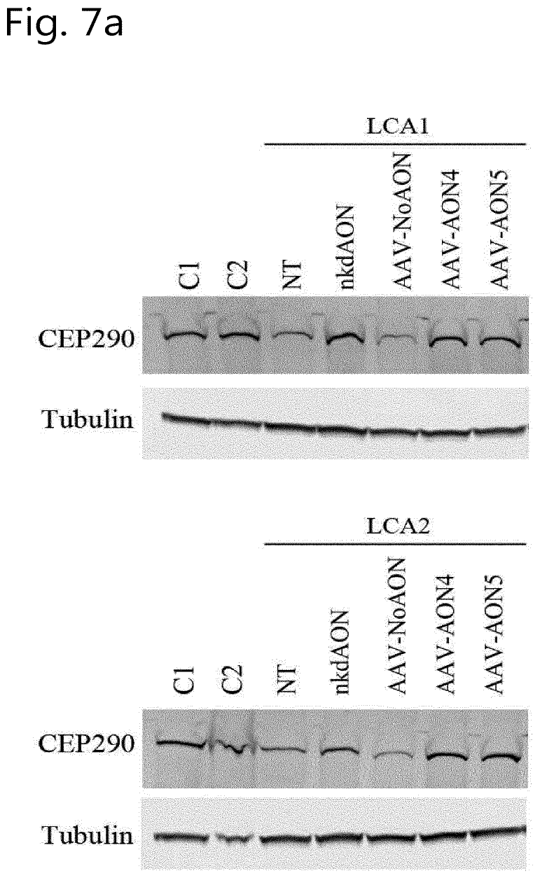

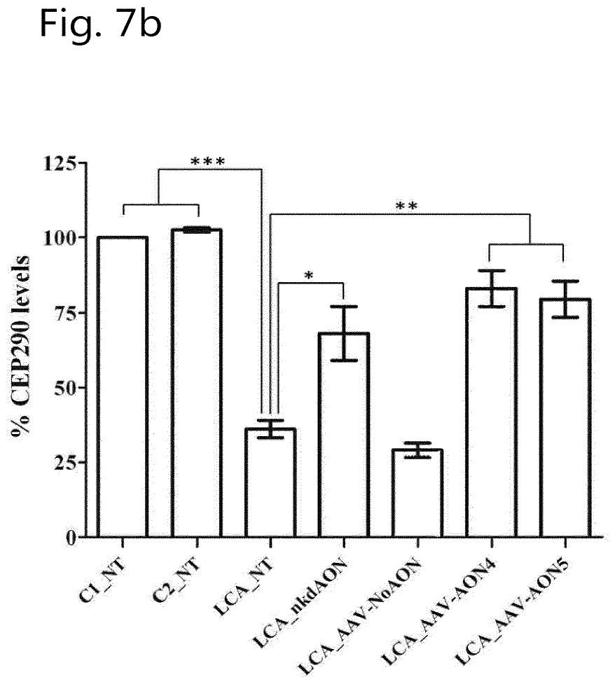

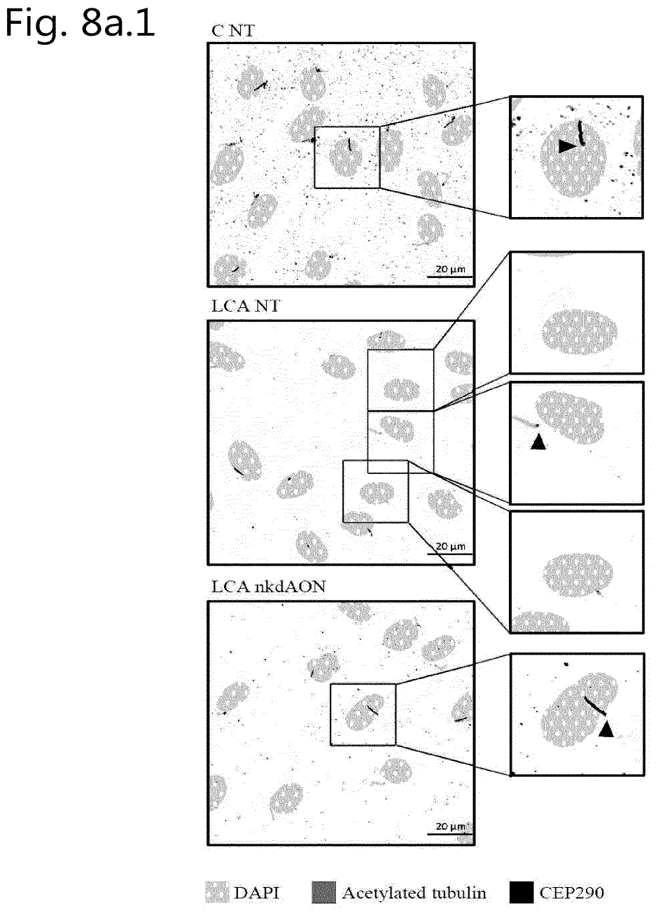

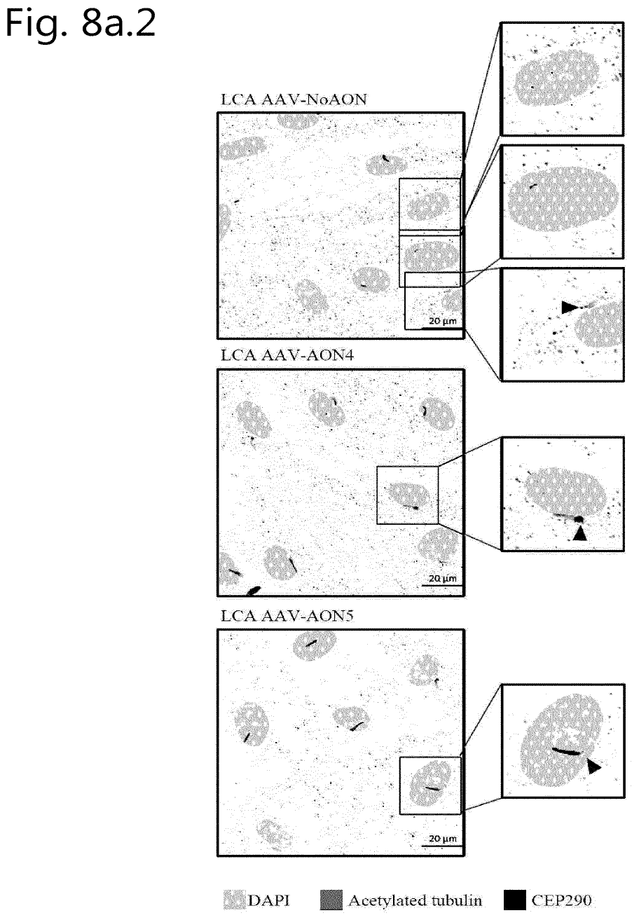

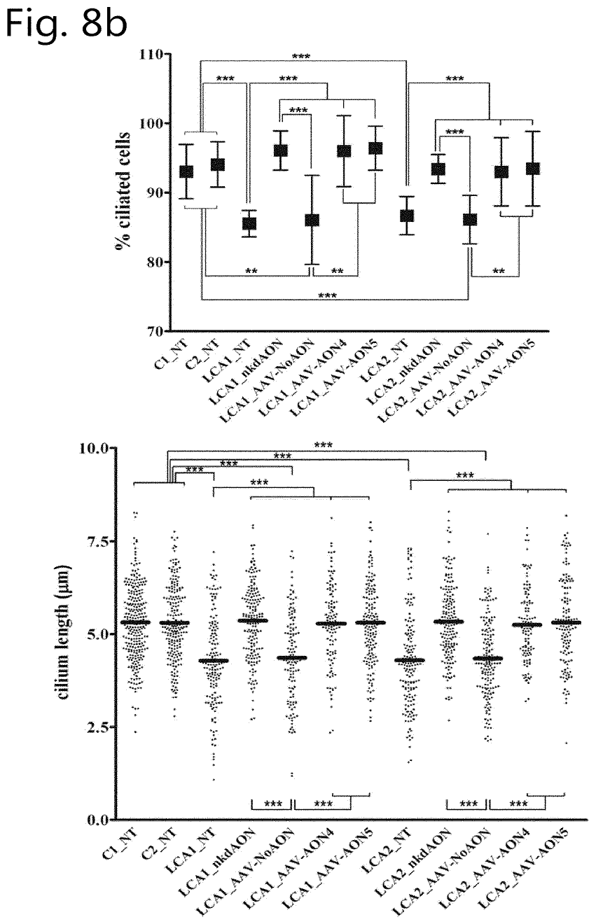

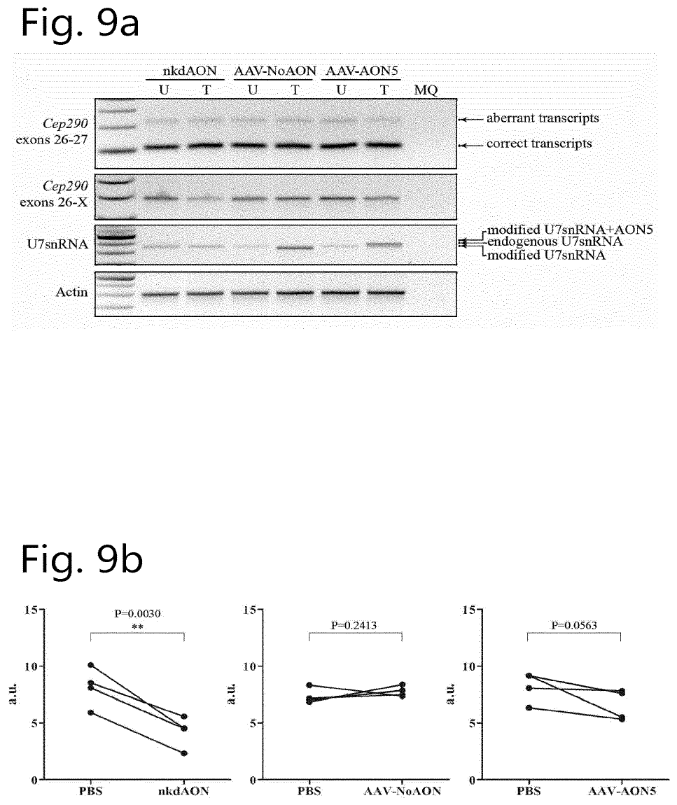

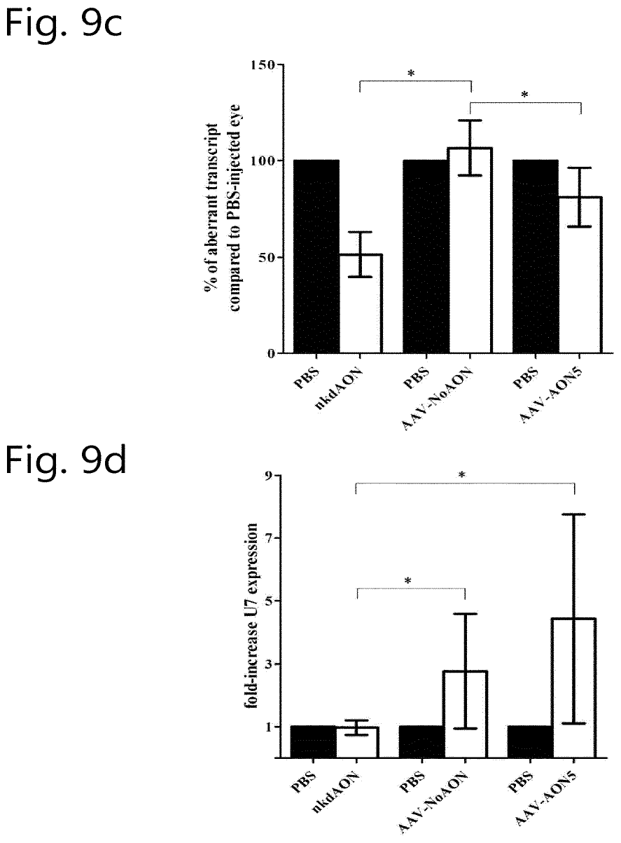

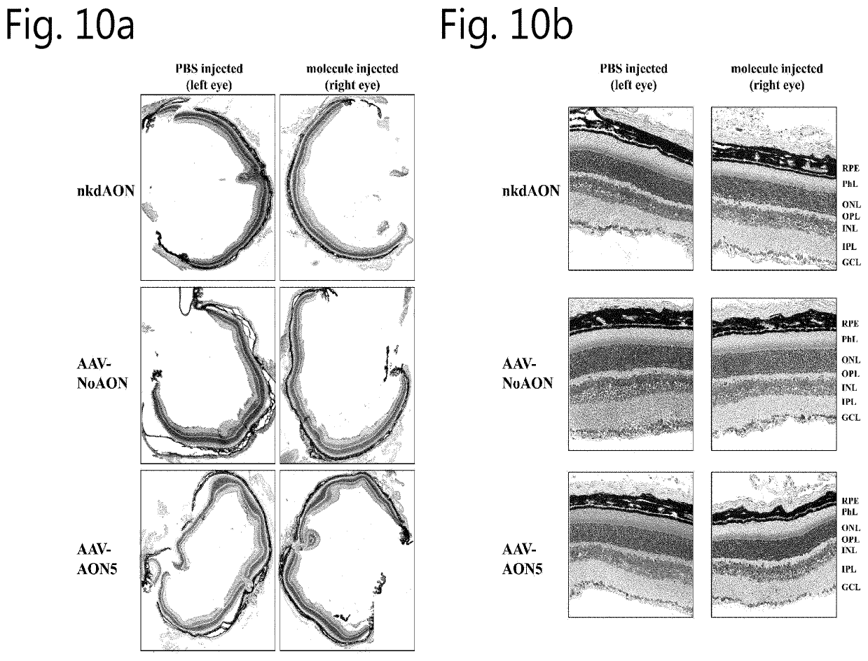

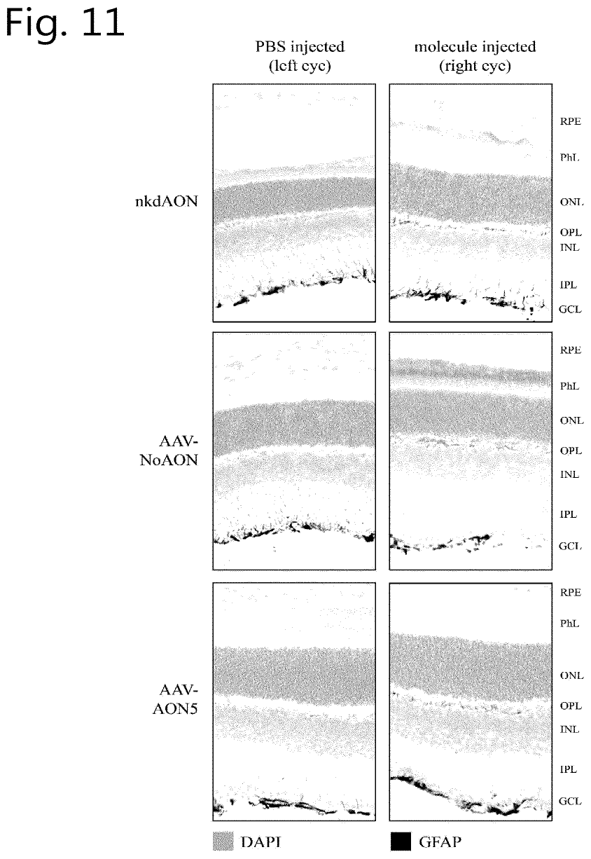

The intronic CEP290 mutation (c.2991+1655A>G) creates a cryptic splice donor site that results in the inclusion of an aberrant exon into the CEP290 mRNA (FIGS. 1A and -B). Addition of AONs directed against the aberrant exon would prevent the insertion of this exon by preventing the binding of factors that are essential for splicing such as the U1- and U2snRNP complexes, and serine-arginine rich proteins, thereby restoring normal CEP290 splicing and protein synthesis (FIG. 1C). AONs can target splice sites as well as exonic sequences, although in the particular case of the Duchenne muscular dystrophy DMD gene, AONs targeting exonic regions tend to outperform those that target the splice sites (Aartsma-Rus et al, 2010). In addition, previous studies have suggested a positive correlation between the capability of AONs to induce exon skipping and the presence of predicted SC35 splice factor binding sites in the target sequence (Aartsma-Rus et al, 2008). To design an AON with high exon-skipping potential, the aberrant CEP290 exon (128 nucleotides exonic sequence plus 15 nucleotides of intronic sequence on each side) was scrutinized for exonic splice enhancer binding motifs, using the ESE finder 3.0 program (Smith et al, 2006). At the 3′-end of the aberrant exon, two SC35-binding motifs were predicted (data not shown). Hence, the first AON was designed such that it encompassed these two motifs (designated AON-3, SEQ ID NO: 11), and being complementary to the CEP290 mRNA.

To determine whether AON-3 has exon-skipping potential in vitro, immortalized lympoblastoid cells of two unrelated individuals with LCA homozygously carrying the intronic CEP290 founder mutation c.2991+1655A>G, as well as one control individual were cultured in the absence or presence of 1 μM AON-3. As expected, in the control individual, only a band representing correctly spliced CEP290 was observed, whereas in both affected individuals two products were present, one representing correctly spliced, and one representing aberrantly spliced CEP290 mRNA. Upon addition of AON-3, a strong decrease in aberrantly spliced CEP290 was noted, in both individuals with LCA (FIG. 2A). Next, the specificity of AON-3 was assessed by transfecting a sense oligonucleotide directed to the same target site (SON-3, SEQ ID NO: 14). RT-PCR analysis showed that in the cells transfected with SON-3, both the aberrantly spliced and the correctly spliced CEP290 mRNA molecules are still present (FIG. 2B, left panel), demonstrating the specificity of the antisense sequence. Using an additional pair of primers that amplifies larger products, similar results were obtained (FIG. 2B, right panel). Interestingly, the decrease in aberrantly spliced CEP290 appears to coincide with an increased intensity of the product representing correctly spliced CEP290 mRNA. These data indicate that the aberrant product is not degraded, but that the AON transfection truly induces exon skipping, resulting in the synthesis of more correctly spliced wild-type CEP290 mRNA. To determine the effective dose of AON-3, cells were transfected with various concentrations of AON-3, ranging from 0.01 to 1.0 μM. Even at the lowest concentration of 0.01 μM, a marked reduction in aberrantly spliced CEP290 was observed. The maximum amount of exon skipping was observed at 0.05 or 0.1 μM of AON, indicating that these concentrations are sufficient to convert almost all aberrantly spliced CEP290 (FIG. 2C).

The effectiveness of AONs in splice modulation is thought to merely depend on the accessibility of the target mRNA molecule, and hence may differ tremendously between neighboring sequences. To determine whether this sequence specificity also applies for CEP290, several AONs were designed that target the aberrant CEP290 exon (Table 1). This exon consists of 128 base pairs, the majority of which are part of an Alu repeat, one of the most frequent repetitive elements in the human genome (Schmidt et al, 1982), covering the entire 5′-end of the aberrant exon (FIG. 3A). Hence, the majority of AONs were designed to be complementary to the 3′-end of the aberrant exon or the splice donor site (FIG. 3A). In total, five AONs were transfected at a final concentration of 0.1 μM, which was shown to be optimal for AON-3. Interestingly, besides AON-3, also AON-2 (SEQ ID NO: 10) and AON-4 (SEQ ID NO: 12) resulted in high levels of exon skipping. In contrast, AON-1 (SEQ ID NO: 9) that targets the Alu repeat region, and AON-5 (SEQ ID NO: 13) that is directed against the splice donor site, hardly showed any exon skipping potential (FIG. 3B). These data demonstrate the sequence specificity in AON-based exon skipping of CEP290 and highlight a small region of the aberrant CEP290 exon as a potential therapeutic target.

### Discussion

In this study, we explored the therapeutic potential of AONs to correct a splice defect caused by an intronic mutation in CEP290. In immortalized lymphoblast cells of LCA patients homozygously carrying the intronic CEP290 mutation c.2991+1655A>G, transfection of some but not all AONs resulted in skipping of the aberrant exon, thereby almost fully restoring normal CEP290 splicing.

AONs have been the focus of therapeutic research for over a decade, for the treatment of a variety of genetic diseases (Hammond et al, 2011). These strategies include the use of AONs to block the recognition of aberrant splice sites, to alter the ratio between two naturally occurring splice isoforms, to induce skipping of exons that contain protein-truncating mutations, or to induce the skipping of exons in order to restore the reading-frame of a gene that is disrupted by a genomic deletion, allowing the synthesis of a (partially) functional protein (Hammond et al, 2011). The latter approach is already being applied in phase I/II clinical trials for the treatment of patients with Duchenne muscular dystrophy, with promising results (Kinali et al, 2009; van Deutekom et al, 2007).

The intronic CEP290 mutation is an ideal target for AON-based therapy, since this mutation results in the inclusion of an aberrant exon in the CEP290 mRNA which is normally not transcribed. Inducing skipping of this aberrant exon by AONs fully restores the normal CEP290 mRNA, allowing normal levels of CEP290 protein to be synthesized.

A second major advantage is that although this AON-approach is a mutation-specific therapeutic strategy, the intronic CEP290 mutation is by far the most frequent LCA-causing mutation.⁴ Based on the estimated prevalence of LCA (1:50,000), and the observed frequency of the intronic CEP290 mutation in Northern-Europe (26%) (Coppieters et al, 2010) and the U.S. (10%) (Stone, 2007), at least one thousand and, depending on the frequency of the mutation in other populations, perhaps many more individuals worldwide have LCA due to this mutation. Finally, although the LCA phenotype associated with CEP290 mutations is severe, it appears that the photoreceptor integrity, especially in the macula, as well as the anatomical structure of the visual connections to the brain, are relatively intact in LCA patients with CEP290 mutations, which would allow a window of opportunity for therapeutic intervention (Cideciyan et al, 2007).

The study described here provides a proof-of-principle of AON-based therapy for CEP290-associated LCA in vitro, using immortalized patient lymphoblast cells. In order to determine the true therapeutic potential of this method for treating LCA, additional studies are needed that include the development of therapeutic vectors, and assessment of efficacy and safety in animal models. Although naked AONs, or conjugated to cell-penetrating peptides, can be delivered to the retina by intraocular injections, the limited stability of the AONs would require multiple injections in each individual. In contrast, by using viral vectors, a single subretinal injection would suffice to allow a long-term expression of the therapeutic construct. Previously, others have used recombinant adeno-associated viral (rAAV) vectors carrying U1- or modified U7snRNA constructs to efficiently deliver AON sequences, in the mdx mouse model for DMD, or in DMD patient myoblasts, respectively (Geib et al, 2009; Goyenhalle et al, 2004). In line with this, AONs targeting the aberrant exon of CEP290 could be cloned within such constructs, and delivered to the retina by subretinal injections of rAAV-5 or -8 serotypes that efficiently transduce photoreceptor cells where the endogenous CEP290 gene is expressed (Alloca et al, 2007; Lebherz et al, 2008). Using rAAV-2 vectors, no long-lasting immune response was evoked upon subretinal injections of these vectors in patients with RPE65 mutations (Simonella et al, 2009), and also for rAAV-5 and rAAV-8, immune responses appear to be absent or limited, at least in animal models (Li et al, 2009; Vandenberghe et al, 2011). One final safety aspect concerns the specificity of the sequence that is used to block the splicing of the aberrant CEP290 exon. As stated before, the majority of this exon is part of an Alu repeat, and AONs directed against this repeat will likely bind at multiple sites in the human genome, increasing the chance to induce off-target effects. The AONs that were shown to be effective in this study do not fully target the Alu repeat sequence, but are also not completely unique in the human genome. However, when blasting against the EST database, no exact hits are found, indicating that at the level of expressed genes, these sequences are unlikely to induce off-target effects and deregulate normal splicing of other genes. To further study the efficacy and safety of AON-based therapy for CEP290-associated LCA in vivo, we are currently generating a transgenic knock-in mouse model that carries part of the human CEP290 gene (exon 26 to exon 27, with and without the intronic mutation) which is exchanged with its mouse counterpart.

Compared to gene augmentation therapy, AON-based therapy has a number of advantages. First, in gene augmentation therapy, a ubiquitous or tissue-specific promoter is used to drive expression of the wild-type cDNA encoding the protein that is mutated in a certain patient. For instance in one clinical trial for RPE65 gene therapy, the chicken beta-actin promoter was used (Maguire et al, 2008). Using these but also fragments of the endogenous promoters, it is difficult to control the levels of expression of the therapeutic gene. In some cases, like for the RPE65 protein that has an enzymatic function, expression levels beyond those of the endogenous gene might not be harmful to the retina. For other genes however, including those that encode structural proteins like CEP290, tightly-regulated expression levels might be crucial for cell survival, and overexpression of the therapeutic protein might exert toxic effects. Using AONs, the therapeutic intervention occurs at the pre-mRNA level, and hence does not interfere with the endogenous expression levels of the target gene. A second issue is the use of the viral vector. Of a variety of different recombinant viral vectors, rAAVs are considered to be most suitable for treating retinal dystrophies, because of their relatively high transduction efficiency of retinal cells, and their limited immunogenicity. The major drawback of rAAVs however is their limited cargo size of 4.8 kb. Again, for some genes like RPE65, this is not a problem. For many other retinal genes however, like CEP290 (with an open reading frame of 7.4 kb), but also ABCA4 and USH2A, the size of their full-length cDNAs exceeds the cargo size of the currently available pool of rAAVs. One way to overcome this problem is to express cDNAs that express only partial proteins with residual activity, as has been suggested for CEP290 by expressing the N-terminal region of CEP290 in a zebrafish model (Baye et al, 2011). Other viral vectors, like lentivirus or adenoviruses have a higher cargo capacity that rAAVs (˜8 kb), but are less efficient in transducing retinal cells, and adenoviruses have a higher immunogenic potential (den Hollander et al, 2010). For AON-based therapy, the size limitations of AAV are not a problem, since the small size of the AONs and the accompanying constructs easily fit within the available AAVs.

In conclusion, this study shows that administration of AONs to cultured patient cells almost fully corrects a splice defect that is caused by a frequent intronic mutation in CEP290 that causes LCA. These data warrant further research to determine the therapeutic potential of AON-based therapy for CEP290-associated LCA, in order to delay or cease the progression of this devastating blinding disease.

### REFERENCE LIST DESCRIPTION AND EXAMPLE 1

1. Leber, T. (1869). Uber Retinitis Pigmentosa and angeborene Amaurose. von Graefe's Archives Ophthalmology 15, 1-25.
2. Koenekoop, R. K., Lopez, I., den Hollander, A. I., Allikmets, R., and Cremers, F. P. (2007). Genetic testing for retinal dystrophies and dysfunctions: benefits, dilemmas and solutions. Clin Experiment Ophthalmol 35, 473-485.
3. Stone, E. M. (2007). Leber congenital amaurosis—a model for efficient genetic testing of heterogeneous disorders: LXIV Edward Jackson Memorial Lecture. Am J Ophthalmol 144, 791-811.
4. den Hollander, A. I., Roepman, R., Koenekoop, R. K., and Cremers, F. P. M. (2008). Leber congenital amaurosis: genes, proteins and disease mechanisms. Prog Retin Eye Res 27, 391-419.
5. Estrada-Cuzcano, A., Koenekoop, R. K., Coppieters, F., Kohl, S., Lopez, I., Collin, R. W. J., De Baere, E. B., Roeleveld, D., Marek, J., Bernd, A. et al (2011). IQCB1 mutations in patients with leber congenital amaurosis. Invest Ophthalmol Vis Sci 52, 834-839.
6. den Hollander, A. I., Koenekoop, R. K., Yzer, S., Lopez, I., Arends, M. L., Voesenek, K. E., Zonneveld, M. N., Strom, T. M., Meitinger, T., Brunner, H. G. et al (2006). Mutations in the CEP290 (NPHP6) gene are a frequent cause of Leber congenital amaurosis. Am J Hum Genet 79, 556-561.
7. Perrault, I., Delphin, N., Hanein, S., Gerber, S., Dufier, J. L., Roche, O., foort-Dhellemmes, S., Dollfus, H., Fazzi, E., Munnich, A. et al (2007). Spectrum of NPHP6/CEP290 mutations in Leber congenital amaurosis and delineation of the associated phenotype. Hum Mutat 28, 416.
8. Baala, L., Audollent, S., Martinovic, J., Ozilou, C., Babron, M. C., Sivanandamoorthy, S., Saunier, S., Salomon, R., Gonzales, M., Rattenberry, E. et al (2007). Pleiotropic effects of CEP290 (NPHP6) mutations extend to Meckel syndrome. Am J Hum Genet 81, 170-179.
9. Frank, V., den Hollander, A. I., Bruchle, N. O., Zonneveld, M. N., Nurnberg, G., Becker, C., Du, B. G., Kendziorra, H., Roosing, S., Senderek, J. et al (2008). Mutations of the CEP290 gene encoding a centrosomal protein cause Meckel-Gruber syndrome. Hum Mutat 29, 45-52.
10. Helou, J., Otto, E. A., Attanasio, M., Allen, S. J., Parisi, M. A., Glass, I., Utsch, B., Hashmi, S., Fazzi, E., Omran, H. et al (2007). Mutation analysis of NPHP6/CEP290 in patients with Joubert syndrome and Senior-Loken syndrome. J Med Genet 44, 657-663.
11. Valente, E. M., Silhavy, J. L., Brancati, F., Barrano, G., Krishnaswami, S. R., Castori, M., Lancaster, M. A., Boltshauser, E., Boccone, L., Al-Gazali, L. et al (2006). Mutations in CEP290, which encodes a centrosomal protein, cause pleiotropic forms of Joubert syndrome. Nat Genet 38, 623-625.
12. Coppieters, F., Casteels, I., Meire, F., De Jaegere S., Hooghe, S., van Regemorter N., Van Esch H., Matuleviciene, A., Nunes, L., Meersschaut, V. et al (2010). Genetic screening of LCA in Belgium: predominance of CEP290 and identification of potential modifier alleles in AHI1 of CEP290-related phenotypes. Hum Mutat 31, E1709-E1766.
13. Littink, K. W., Pott, J. W., Collin, R. W. J., Kroes, H. Y., Verheij, J. B., Blokland, E. A., de Castro Miro M., Hoyng, C. B., Klaver, C. C., Koenekoop, R. K. et al (2010). A novel nonsense mutation in CEP290 induces exon skipping and leads to a relatively mild retinal phenotype. Invest Ophthalmol Vis Sci 51, 3646-3652.
14. Bainbridge, J. W., Smith, A. J., Barker, S. S., Robbie, S., Henderson, R., Balaggan, K., Viswanathan, A., Holder, G. E., Stockman, A., Tyler, N. et al (2008). Effect of gene therapy on visual function in Leber's congenital amaurosis. N Engl J Med 358, 2231-2239.
15. Cideciyan, A. V., Aleman, T. S., Boye, S. L., Schwartz, S. B., Kaushal, S., Roman, A. J., Pang, J. J., Sumaroka, A., Windsor, E. A., Wilson, J. M. et al (2008). Human gene therapy for RPE65 isomerase deficiency activates the retinoid cycle of vision but with slow rod kinetics. Proc Natl Acad Sci USA 105, 15112-15117.
16. Hauswirth, W., Aleman, T. S., Kaushal, S., Cideciyan, A. V., Schwartz, S. B., Wang, L., Conlon, T., Boye, S. L., Flotte, T. R., Byrne, B. et al (2008). Phase I Trial of Leber Congenital Amaurosis due to Estrada-Mutations by Ocular Subretinal Injection of Adeno-Associated Virus Gene Vector: Short-Term Results. Hum Gene Ther 17. Maguire, A. M., Simonelli, F., Pierce, E. A., Pugh, E. N., Jr., Mingozzi, F., Bennicelli, J., Banfi, S., Marshall, K. A., Testa, F., Surace, E. M. et al (2008). Safety and efficacy of gene transfer for Leber's congenital amaurosis. N Engl J Med 358, 2240-2248.
18. Maguire, A. M., High, K. A., Auricchio, A., Wright, J. F., Pierce, E. A., Testa, F., Mingozzi, F., Bennicelli, J. L., Ying, G. S., Rossi, S. et al (2009). Age-dependent effects of RPE65 gene therapy for Leber's congenital amaurosis: a phase 1 dose-escalation trial. Lancet 374, 1597-1605.
19. den Hollander, A. I., Black, A., Bennett, J., and Cremers, F. P. M. (2010).
Lighting a candle in the dark: advances in genetics and gene therapy of recessive retinal dystrophies. J Clin Invest 120, 3042-3053.
20. Aartsma-Rus, A., Houlleberghs, H., van Deutekom, J. C., van Ommen, G. J., and 't Hoen, P. A. (2010). Exonic sequences provide better targets for antisense oligonucleotides than splice site sequences in the modulation of Duchenne muscular dystrophy splicing. Oligonucleotides 20, 69-77.
21. Aartsma-Rus, A., van, V. L., Hirschi, M., Janson, A. A., Heemskerk, H., de Winter, C. L., de, K. S., van Deutekom, J. C., 't Hoen, P. A., and van Ommen, G. J. (2008). Guidelines for Antisense Oligonucleotide Design and Insight Into Splice-modulating Mechanisms. Mol Ther
22. Smith, P. J., Zhang, C., Wang, J., Chew, S. L., Zhang, M. Q., and Krainer, A. R. (2006). An increased specificity score matrix for the prediction of SF2/ASF-specific exonic splicing enhancers. Hum Mol Genet 15, 2490-2508.
23. Schmid, C. W. and Jelinek, W. R. (1982). The Alu family of dispersed repetitive sequences. Science 216, 1065-1070.
24. Hammond, S. M. and Wood, M. J. (2011). Genetic therapies for RNA mis-splicing diseases. Trends Genet 27, 196-205.
25. Kinali, M., rechavala-Gomeza, V., Feng, L., Cirak, S., Hunt, D., Adkin, C., Guglieri, M., Ashton, E., Abbs, S., Nihoyannopoulos, P. et al (2009). Local restoration of dystrophin expression with the morpholino oligomer AVI-4658 in Duchenne muscular dystrophy: a single-blind, placebo-controlled, dose-escalation, proof-of-concept study. Lancet Neurol 8, 918-928.
26. van Deutekom, J. C., Janson, A. A., Ginjaar, L B., Frankhuizen, W. S., Aartsma-Rus, A., Bremmer-Bout, M., Den Dunnen, J. T., Koop, K., van der Kooi, A. J., Goemans, N. M. et al (2007). Local dystrophin restoration with antisense oligonucleotide PRO051. N Engl J Med 357, 2677-2686.
27. Coppieters, F., Lefever, S., Leroy, B. P., and De, B. E. (2010). CEP290, a gene with many faces: mutation overview and presentation of CEP290base. Hum Mutat 31, 1097-1108.
28. Cideciyan, A. V., Aleman, T. S., Jacobson, S. G., Khanna, H., Sumaroka, A., Aguirre, G. K., Schwartz, S. B., Windsor, E. A., He, S., Chang, B. et al (2007). Centrosomal-ciliary gene CEP290/NPHP6 mutations result in blindness with unexpected sparing of photoreceptors and visual brain: implications for therapy of Leber congenital amaurosis. Hum Mutat 28, 1074-1083.
29. Geib, T. and Hertel, K. J. (2009). Restoration of full-length SMN promoted by adenoviral vectors expressing RNA antisense oligonucleotides embedded in U7 snRNAs. PLoS One 4, e8204.
30. Goyenvalle, A., Vulin, A., Fougerousse, F., Leturcq, F., Kaplan, J. C., Garcia, L., and Danos, O. (2004). Rescue of dystrophic muscle through U7 snRNA-mediated exon skipping. Science 306, 1796-1799.
31. Allocca, M., Mussolino, C., Garcia-Hoyos, M., Sanges, D., Iodice, C., Petrillo, M., Vandenberghe, L. H., Wilson, J. M., Mango, V., Surace, E. M. et al (2007). Novel adeno-associated virus serotypes efficiently transduce murine photoreceptors. J Virol 81, 11372-11380.
32. Lebherz, C., Maguire, A., Tang, W., Bennett, J., and Wilson, J. M. (2008). Novel AAV serotypes for improved ocular gene transfer. J Gene Med 10, 375-382.
33. Simonelli, F., Maguire, A. M., Testa, F., Pierce, E. A., Mingozzi, F., Bennicelli, J. L., Rossi, S., Marshall, K., Banfi, S., Surace, E. M. et al (2009). Gene Therapy for Leber's Congenital Amaurosis is Safe and Effective Through 1.5 Years After Vector Administration. Mol Ther
34. Li, W., Kong, F., Li, X., Dai, X., Liu, X., Zheng, Q., Wu, R., Zhou, X., Lu, F., Chang, B. et al (2009). Gene therapy following subretinal AAV5 vector delivery is not affected by a previous intravitreal AAV5 vector administration in the partner eye. Mol Vis 15, 267-275.
35. Vandenberghe, L. H., Bell, P., Maguire, A. M., Cearley, C. N., Xiao, R., Calcedo, R., Wang, L., Castle, M. J., Maguire, A. C., Grant, R. et al (2011). Dosage Thresholds for AAV2 and AAV8 Photoreceptor Gene Therapy in Monkey. Sci Transl Med 3, 88ra54.
36. Baye, L. M., Patrinostro, X., Swaminathan, S., Beck, J. S., Zhang, Y., Stone, E. M., Sheffield, V. C., and Slusarski, D. C. (2011). The N-terminal region of centrosomal protein 290 (CEP290) restores vision in a zebrafish model of human blindness. Hum Mol Genet 20, 1467-1477.
37. Dom and Kippenberger, Curr Opin Mol Ther 2008 10(1) 10-20
38. Nielsen, et al. (1991) Science 254, 1497-1500
39. Govindaraju and Kumar (2005) Chem. Commun, 495-497
40. Egholm et al (1993) Nature 365, 566-568
41. Morita et al. 2001. Nucleic Acid Res Supplement No. 1: 241-242
42. Gorman L, et al, Stable alteration of pre-mRNA splicing patterns by modified U7 small nuclear RNAs. Proc Natl Acad Sci USA 1998; 95(9):4929-34
43. Suter D, et al, Double-target antisense U7 snRNAs promote efficient skipping of an aberrant exon in three human beta-thalassemic mutations. Hum Mol Genet 1999; 8(13):2415-23
44. Remington: The Science and Practice of Pharmacy, 20th Edition. Baltimore, Md.: Lippincott Williams & Wilkins, 2000

### Example 2: AON's Delivered by Adeno Associated Viral Vectors

### Abstract

Leber congenital amaurosis (LCA) is a genetically heterogeneous disorder characterized by severe visual impairment starting in the first year of life. The most frequent genetic cause of LCA, present in up to 15% of all LCA cases in some Western populations, is an intronic mutation in CEP290 (c.2991+1655A>G) that creates a cryptic slice donor site and results in the insertion of an aberrant exon into CEP290 mRNA. In example 1, we have shown that antisense oligonucleotides (AONs) effectively restore normal CEP290 splicing in patient-derived lymphoblastoid cells. Given the safety and efficacy of adeno-associated viruses (AAVs) used in ongoing clinical trials for other genetic subtypes of retinal dystrophy, we here aimed to explore the therapeutic potential of AAV-based delivery of AONs. Transduction of patient-derived fibroblast cells with effective AONs cloned into a modified U7snRNA construct and packaged into AAV2/2 fully restored normal CEP290 pre-mRNA splicing and significantly increased CEP290 protein levels. Moreover, a ciliary phenotype present in these fibroblasts was completely rescued upon transduction of AON-containing AAVs. Together, our data show that AAVs are an excellent therapeutic vector for the delivery of AONs to restore splice defects, and highlight the tremendous potential of AONs for the treatment of CEP290-associated LCA.

### Introduction

Leber congenital amaurosis (LCA; OMIM 204000) is a group of early-onset, rare and severe inherited retinal dystrophies (IRDs) with a prevalence of 1:50,000 in the European and North-American populations (1, 2). The clinical characteristics of LCA are severe and early loss of vision, amaurotic pupils, sensory nystagmus and the absence of electrical signals on electroretinogram (ERG) (3). LCA shows a high genetic heterogeneity and to date 20 different genes have been associated with LCA (sph.uth.edu/retnet), mainly segregating in an autosomal recessive manner. The most frequently mutated LCA gene is CEP290 (centrosomal protein 290 kDa) (3-5), a gene that encompasses 54 exons and encodes a 2479 amino acid protein (6) localized in the centrosome and in the basal body of cilia (7). Of all CEP290 mutations that cause non-syndromic LCA, a deep-intronic variant (c.2991+1655A>G) is by far the most recurrent one, accounting for up to 15% of LCA cases in many Western countries (2, 5, 8, 9). This mutation creates a cryptic splice donor site, resulting in the insertion of an aberrant exon with a premature stop codon into ˜50% of all CEP290 transcripts (5).

For many years, retinal dystrophies (RDs) have been considered incurable diseases. However, in the last decade major progress has been accomplished, mainly in the field of gene therapy. Phase I/II clinical trials using gene augmentation therapy have shown to be safe and moderately effective in LCA and early-onset RD patients with mutations in RPE65 (10-13), and in choroideremia patients with a mutation in CHM (14). In these studies, the wild-type cDNA of RPE65 and CHM, respectively, was packaged in replication-defective recombinant adeno-associated viruses (AAVs) and delivered to the retina by a single surgical procedure. The restricted cargo size of AAVs (˜5 kb) however has so far hampered a fast and broad implementation of AAV-based gene augmentation therapy for the many other genetic subtypes of IRD, since the cDNA size of many genes that cause IRD, including that of CEP290, way exceed the cargo limit of AAVs.

One alternative strategy to treat CEP290-associated LCA utilizes antisense oligonucleotides (AONs), small RNA molecules that complementary bind to its target mRNA and subsequently can interfere with pre-mRNA splicing. AONs have already been shown to be effective therapeutic molecules in several inherited disorders in vitro (15) and are currently being used in clinical trials for Duchenne's muscular dystrophy (16, 17), several cancer types (18, 19), familial hypercholesterolemia (20), viral infections or neovascular disorders (21) amongst others. The intronic CEP290 mutation is an ideal target for AON-based therapy, as skipping the cryptic exon that is inserted to the CEP290 mRNA as a result of the c.2991+1655A>G change, would fully restore normal CEP290 splicing and restore wild-type CEP290 protein levels. Recently, we and others have shown that transfection of naked AON molecules indeed restores normal CEP290 splicing in cultured cells of LCA patients homozygously carrying the intronic CEP290 mutation (22, 23). In future trials however, administrating AONs to the retina of patients with CEP290-associated LCA would require multiple injections of naked AONs, as the stability of these molecules depends among others on chemical modifications, target cell or tissue, and ranges from hours to months (21, 24). An alternative way of delivering AONs would be to use AAVs, as a single subretinal injection of AAV could give rise to a persistent expression, potentially life long, as shown in dog and primate models (25, 26). Hence, we here investigated the therapeutic efficacy of AONs that are cloned into AAVs, and provide evidence that AAV-based delivery of AONs is an excellent approach to treat CEP290-associated LCA.

### Material and Methods

### Study Design

The objective of this work is to assess the efficacy of AAV-mediated AON-based therapy for CEP290-associated LCA. Fibroblast cell lines derived from two unrelated LCA patients homozygously carrying the c.2991+1655A>G mutation, and from two age- and gender matched healthy individuals were used. Outcome measures include the correction of aberrant CEP290 splicing via RT-PCR, the assessment of CEP290 protein levels via Western blot analysis, and a qualitative and quantitative characterization of cilium structure via immunofluorescence microscopy. All experiments were performed simultaneously in all cell lines.

### Ethics Statement

Our research was conducted according to the tenets of the Declaration of Helsinki. The procedures for obtaining human skin biopsies to establish primary fibroblasts cell lines were approved by the Ethical Committee of the Radboud University Medical Centre (Commissie Mensgebonden Onderzoek Arnhem-Nijmegen). Written informed consent was gathered from all participating individuals by signing the Declaration of Permission for the Use of Body Material (Toestemmingsverklaring gebruik lichaamsmateriaal) of the Radboud University Medical Centre. All procedures were carried out in the Netherlands.

### Construct Design

AON sequences were cloned into a modified U7snRNA gene in the pSMD2 shuttle vector that contains the inverted terminal repeat (ITR) sequences for AAV production, via a two-step PCR approach as described elsewhere (27). This yielded six different AON-containing vectors and one control vector (FIG. 4A), coined pAAV-AON1 to pAAV-AON6.

Previously, we cloned a ˜6.4 kb fragment of the CEP290 gene that contained part of intron 25, exon 26, intron 26 (either with or without the intronic mutation), exon 27 and part of intron 27 (28), and inserted into the pCI-Neo plasmid flanked by the exon 3 and 5 of the Rhodopsin gene (29). These constructs are referred to as WT and LCA minigene constructs.

### Cell Culture and Transfection

Fibroblast cell lines derived from skin biopsies of individuals with CEP290-associated LCA or healthy controls were cultured in DMEM, supplemented with 20% fetal bovine serum (FBS), 1% penicillin-streptomycin and 1% of sodium pyruvate at 37° C. and 5% CO₂. HEK293T cells were cultured in DMEM supplemented with 10% FBS, 1% penicillin-streptomycin and 1% sodium pyruvate at 37° C. and 5% CO₂.

In order to validate the effectiveness of the AON-containing vectors, serial dilutions were carried out by co-transfecting 1 μg of the LCA minigene construct, together with various amounts of each pAAV-AON vector (0.5 μg; 0.125 μg and 0.035 μg) in human embryonic kidney (HEK293T) cells. Co-transfections were performed by combining the two plasmids with FuGene (Promega, Madison, Wis.) reagent (1:3 ratio) following manufacturer's protocol. Naked AON (0.1 μmol/l) was used as a positive control. Cells were harvested for transcriptional analysis 48 h post-transfection.

### AON-Containing AAV Generation

The two most effective AONs were selected for AAV2/2 production. Plasmid DNA was purified by the Megaprep kit (Qiagen, Venlo, the Netherlands). The U7snRNA-AON constructs were packaged into AAV by transfection of three plasmids (AAV pSMD2 plasmid containing the U7snRNA-AON, AAV package plasmid encoding AAV Rep and Cap proteins from serotype 2 and adenovirus helper plasmid) in HEK 293 cells. Three days after transfection, cells and culture medium were collected and enzymatically treated with Benzonase and a high salt solution. The cell debris was removed by high speed centrifugation and regular filtration. The supernatant went through a tangential flow filter which concentrated the viral solution. Recombinant AAV vector particles were isolated and extracted by running the concentrated supernatant through the iodixanol density gradient. The purified supernatant was then further concentrated by running through an Amicon filter with a 100,000 molecular weight cut off. The pure AAVs were then tittered by real-time PCR and the purity was verified by SDS page gel electrophoresis. For assessing which AAV serotype most effectively transduces fibroblast cells, an existing panel of AAVs containing an expression cassette of GFP under control of the cytomegalovirus (CMV) promoter was used.

### AAV Transduction in Fibroblasts

Fibroblast cell lines were transfected or transduced as follow. For transfection, cells were seeded in the corresponding plate according to the amount of cells needed and, transfected with 0.1 μmol of naked AON using FuGene HD (Promega, Madison, Wis.) as described before. For transduction, cells were transduced with AAV at different multiplicities of infection (MOIs) (100; 1,000 and 10,000) and medium was replaced after 24 h. Cells were harvested 96 h later for transcriptional analysis. For protein and immunocytochemistry studies cells were transduced with a MOI of 10,000 or transfected with 0.1 μmol of naked AON. For protein analysis 1,800,000 cells were seeded in 10 cm dishes and harvested 96 h following transfection/transduction. For immunocytochemistry, fibroblast cells were seeded on coverslips in a 12-well plate and 48 h following transfection/transduction, medium was replaced for a low FBS (0.2%) containing medium for another 48 h.

### RNA Isolation and RT-PCR Analysis

Fibroblast cells were used for RNA isolation (Nucleospin RNA II, Duren, Germany) following the manufacturer's protocol. Five hundred nanograms of RNA were used for cDNA synthesis by using the iScript cDNA Synthesis kit (Bio-Rad, Hercules, Calif.) at a final volume of 20 μl and then diluted 3.5 times by adding 50 μl of RNAse-free H₂O. All the PCR reaction mixtures (25 μl) contained 10 μM of each primer pair, 2 μM of dNTPs, 1.5 mM MgCl₂, 10% Q-solution (Qiagen, Venlo, Netherlands), 1 U of Taq polymerase (Roche, Penzberg, Germany) and 5 μl of diluted cDNA. PCR conditions were 94° C. for 2 min, followed by 35 cycles of 20 s at 94° C., 30 s at 58° C. and 30 s at 72° C., with a final extension step of 2 min at 72° C. Amplicons were analyzed by agarose electrophoresis. Actin expression was used to compare and normalize samples. For co-transfection of the minigene with the AON vectors, Rhodopsin and U7 primers were used to assess the transfection efficiency. All oligonucleotide sequences are listed in Supplementary Table 1.

### Western Blot Analysis

Fibroblast cells were homogenized in 100 μl of RIPA buffer (50 mM Tris pH 7.5, 1 mM EDTA, 150 mM NaCl, 0.5% Na-Deoxycholate, 1% NP40 plus protease inhibitors). Total protein was quantified using the BCA kit (Thermo Fisher Scientific, Waltham, Mass.). For CEP290 detection, ˜75 μg of total protein lysate supplemented with sample buffer was loaded onto a NuPage 3-8% tris-acetate gel (Life technologies, Carlsbad, Calif.). The electrophoresis was carried out for 4 h at 150 V. For normalization, ˜25 μg of the same protein lysates were loaded onto a NuPage 4-12% bis-acrylamide tris-glycine gel (Life technologies, Carlsbad, Calif.) for detection of α-tubulin and run for 2 h at 150 V. All lysates were boiled for 5 min at 98° C. prior to loading. Proteins were transferred to a PVDF membrane (GE Healthcare, Little Chalfont, UK) overnight at 25 V and 4° C. Blots were blocked in 5% non-fat milk in PBS for 6 h at 4° C., incubated overnight at 4° C. with rabbit anti-CEP290 (dilution 1:750, Novus Biological, Littleton, Colo.) or mouse anti-a-tubulin (dilution 1:2,000, Abcam, Cambridge, UK) in 0.5% non-fat milk in PBS solution, washed in PBST (4×5 min), incubated with the appropriate secondary antibody for 1 h at room temperature (RT), washed in PBST (4×5 min) and developed using the Odissey Imaging System (Li-Cor Biosciences, Lincoln, Nebr.). Semi-quantification was performed using Image J software (30).

### Immunofluorescence Analysis

Cells were grown on coverslips in 12 well plates and transfected with 0.1 μmol of naked AON or transduced with AAVs (MOI of 10,000). After 48 h of serum starvation, cells were rinsed with 1×PBS (137 mM NaCl, 2.7 mM KCl, 1.5 mM KH₂PO₄, and 8 mM Na₂HPO₄, pH 7.4), fixed in 2% paraformaldehyde for 20 min, permeabilized in PBS with 0,1% Triton X for 5 min and blocked for 30 min with 2% BSA in PBS at RT. Primary antibody was diluted in blocking solution and incubated for 90 min at RT. Subsequently, slides were washed 5 min in PBS three times, incubated with 1:500 dilution of the corresponding Alexa Fluor-conjugated antibody (Molecular Probes, Eugene, Oreg.) for 45 min. Finally, slides were washed in PBS 3×5 min and mounted in Vectashield with DAPI (Vector laboratories, Burlingame, Calif.). Primary antibodies dilutions were 1:1,000 for mouse anti-acetylated tubulin (Sigma-Aldrich, St. Louis, Mo.) and 1:300 for rabbit anti-CEP290 (Novus Biological, Littleton, Colo.). Pictures were taken at 40× with Axio Imager (Zeiss, Oberkochen, Germany) microscope. For each condition, at least 150 ciliated cells were counted and their cilia were measured by using Image J software (30).

### Statistical Analysis

In order to study the differences between treated and untreated cells we applied the two-tailed Student's T and Mann-Whitney tests. P-values smaller than 0.05 were considered significant as indicated in the figures. Statistical analysis was performed for the quantification of the CEP290 protein levels, as well as the ciliation and cilium length measurements.

### Results

In this study, we aimed to explore the therapeutic efficacy of AAV-based delivery of antisense oligonucleotides, to correct a splice defect resulting from a recurrent intronic mutation in CEP290. Previously, we showed that delivery of naked AONs restores normal CEP290 splicing in lymphoblastoid cells from LCA patients homozygously carrying the intronic CEP290 mutation (22). To determine the consequences of splice correction at the protein and cellular level, fibroblast cell lines were generated from LCA patients with the intronic CEP290 mutation, as these fibroblasts, in contrast to lymphoblastoid cells, endogenously express the CEP290 protein and develop cilia when cultured under serum starved conditions. Similar to what was observed in lymphoblasts (22), transfection of naked AONs to the patient's fibroblast cells completely restored normal CEP290 splicing, with a minimal effective concentration of 0.05 μmol/l (data not shown). In order to assess the efficacy of AAV-mediated AON delivery, the naked AON was used as a positive control in all experiments, at a final concentration of 0.1 μmol/l.

In order to deliver AONs in an adeno-associated viral context, a modified U7snRNA construct was used. This allows the synthesis of the RNA molecules and an effective delivery of AONs to the right nuclear compartment for splicing intervention (27). Different AON sequences (or combinations thereof) were cloned into the pSMD2 vector that contains the modified U7snRNA as well as inverted terminal repeat (ITR) sequences (27) needed for AAV generation (FIG. 4A), yielding six different constructs with AONs (pAAV-AON1 to pAAV-AON6), as well as a construct without any AON that served as a negative control (pAAV-NoAON). However, upon transfection of these constructs into the patient's fibroblasts, no decrease in the amount of aberrantly spliced CEP290 transcript was observed, due to the very low transfection efficiency in this cell type (data not shown). In order to assess the potential therapeutic efficacy of the generated constructs, two CEP290 minigene constructs were generated that contained ˜6.4 kb of the human CEP290 gene, including exon 26, the complete intron 26 (with and without the intronic mutation), and exon 27, flanked by two exons of the RHO gene (FIG. 4B). Transfection of these constructs in human embryonic kidney (HEK293T) cells revealed that the proportion of aberrantly vs. correctly spliced CEP290 transcripts was comparable to that observed in LCA fibroblast cells (FIG. 4C), hence mimicking the molecular consequences of the intronic CEP290 mutation in these cells. Subsequent co-transfection of the CEP290 minigene construct with the six different pAAV-AON constructs into HEK293T cells revealed that all constructs were able to redirect normal CEP290 splicing (FIG. 5). To identify the most potent vectors, decreasing quantities of pAAV-AON constructs were transfected, revealing pAAV-AON4 and pAAV-AONS as the most effective ones, as these were still able to fully restore CEP290 splicing at the lowest concentration tested (FIG. 5). Thus, pAAV-AON4 and pAAV-AONS were selected for the generation of AAVs, together with the pAAV-NoAON construct.

The AAV capsid serotype determines the capability to infect certain cells or tissues. To determine which serotype was most suitable for our experiments, we transduced 6 different AAV serotypes carrying the cDNA of the GFP under control of a CMV promoter. AAV2/2 showed the highest transduction efficiency, already at an MOI of 100, in both control and LCA patient fibroblast cells (data not shown). Higher MOIs determined that AAV2/9 was also able to infect LCA patient fibroblast cells (data not shown), but AAV2/2 was selected for the generation of the AON-containing AAVs.

Following the generation of the three AAVs (i.e. AAV-NoAON, AAV-AON4 and AAV-AON5), the fibroblast cell lines of two unrelated LCA patients homozygously carrying the intronic CEP290 mutation (LCA1 and LCA2) were transduced with these AAVs, at three different multiplicities of infection (MOI), or transfected with the naked AON that served as a positive control. RT-PCR analysis revealed that transduction of the two AON-containing AAVs almost completely restored normal CEP290 splicing, with the highest efficacy observed at an MOI of 10,000 (FIG. 6). In contrast, AAV-NoAON showed the same pattern as the untransduced cells, indicating that the rescue of aberrant CEP290 splicing was caused by the actual AON sequences. Transduction of two control fibroblast cell lines (C1 and C2) with the different AAVs did not show any difference in the levels of expression of CEP290 mRNA (data not shown).

Next, we assessed whether restoring normal CEP290 splicing resulted in an increase of wild-type CEP290 protein levels. Patient and control fibroblast cells were transduced with the three AAVs at an MOI of 10,000, or transfected with the naked AONs. Protein lysates were subjected to Western blot analysis, using α-tubulin as a loading control. Whereas in the untreated fibroblast cells from the LCA patients, CEP290 protein levels were markedly reduced, cells transduced with AAV-AON4 and AAV-AON5 showed a significant increase in CEP290 protein levels, almost reaching those observed in healthy controls (FIGS. 7A and 7B). Again, no differences were observed between the untransduced cells and those transduced with AAV-NoAON (FIGS. 7A and 7B). In addition, we studied the effect of AAV transduction in control fibroblasts, and no change in CEP290 protein levels was observed for any of the conditions tested (data not shown).

CEP290 localizes in the basal body of the cilium and is thought to play an important role in cilium development and/or ciliary transport (7, 31). When cultured under serum starving conditions, fibroblast cells develop cilia (32). When comparing the appearance of cilia in control fibroblasts vs. fibroblasts of LCA patients with the intronic CEP290 mutation, a clear and statistically significant ciliary phenotype was observed, i.e. a reduced number of ciliated cells, and a shorter average length of the cilium (FIG. 8). Remarkably, the fibroblast cells of the LCA patients displayed a high heterogeneity, showing three different appearances: no cilium, a short cilium or a normal cilium (FIGS. 8A1 and 8A2). Only the cells with a normal cilium showed a positive signal for CEP290 staining in the basal body, indicating that the cilium length is directly correlated to a proper expression and localization of the CEP290 protein. In addition, this variability suggests that the 1:1 ratio of the aberrant and normal transcripts is an average of a population of cells, and may differ amongst individual cells. To assess whether the ciliary phenotype could be restored by AONs, fibroblast cells were again transfected (0.1 μmol/l) or transduced (MOI of 10,000) with AONs or AAV-AONs and subjected to serum starvation 48 hours following transfection/transduction. Our results showed that the ciliary phenotype was completely rescued after 96 hour treatment (FIGS. 8A1 and 8A2), i.e. the number of ciliated cells as well as the average length of the cilium returned to the values observed in control fibroblasts (FIG. 8B). Notable, the rescue of the ciliary phenotype was accompanied by a marked increase of CEP290 staining at the base of the cilium, as is apparent from the immunocytochemistry images presented in FIGS. 8A1 and 8A2. No differences in ciliation and cilium length were observed following AON treatment in control cell lines (data not shown).

### Discussion

Mutations in CEP290 are the most common genetic cause of LCA in the Caucasian population, accounting for up to 20% of the cases (3). Intruigingly, an intronic founder mutation in CEP290 (c.2991+1655A>G) on itself explains already 15% of all LCA cases in several Western countries, including the US, France, Belgium and The Netherlands (2, 5, 8, 9), and shows a somewhat lower prevalence in other European countries (33). Therefore, CEP290, and in particular the intronic mutation, has emerged as an attractive target for developing genetic therapies. In this study, we show that AAV-based delivery of antisense oligonucleotides effectively rescues the cellular phenotype associated with the common intronic CEP290 mutation, highlighting the enormous potential of this therapeutic approach.

For several genetic subtypes of IRD, preclinical therapeutic intervention studies are ongoing, with encouraging results in many of these studies (34, 35). In addition, clinical trials employing viral-based gene augmentation therapy have been initiated for five different genetic subtypes of IRD, i.e. caused by mutations in ABCA4, CHM, MERTK, MYO7A and RPE65. Subretinal delivery of AAVs carrying wild-type RPE65 cDNA to the retinal pigment epithelium showed, besides some moderate improvement in visual function (add the same four references as in introduction) a high safety profile, with no risk of insertional mutagenesis, and very low or absent immune responses to the AAV, even upon readministration of the virus in the second eye (36). More recently, AAV-based delivery of CHM cDNA also turned out to be safe and moderately effective in six treated patients with choroideremia (14). A serious constraint of AAVs however is their limited cargo capacity, as only transgenes smaller than 5 kb can be efficiently packaged in these vectors (34). Therefore, many large genes are not eligible for AAV-mediated delivery, as is the case for CEP290 whose coding sequence is ˜7.5 kb long. Recently, dual AAV strategies have been developed that consist of delivering two different AAV vectors, each containing half of the cDNA with a small overlapping region, allowing the assembly of the complete transcript in the target cell following transduction. Although promising results have been obtained in reconstituting full-length cDNAs of ABCA4 and MYO7A in mutant mouse models for Stargardt's disease and Usher syndrome type 1B, respectively (37, 38), not much success has been achieved so far for CEP290 (Renee C. Ryals, personal communication). Alternatively, lentiviral vectors can carry cargos up to 10 kb (34), which would be sufficient for packaging the full-length CEP290 cDNA. A disadvantage of lentiviruses however is that they integrate into the genome of the host cell, running the risk of insertional mutagenesis. In addition, another problem associated with gene augmentation is the fact that expression levels cannot be regulated, therefore increasing the risk of toxicity upon reaching expression levels exceeding those of the endogenous protein. Recently, it was shown that transduction of lentiviruses containing wild-type CEP290 cDNA under control of a CMV promoter could rescue a ciliary phenotype in patient-derived fibroblast cells, although in the same study, toxic effects were observed upon transduction of these lentiviruses in other cell types (39). Together, these studies suggest that there are many challenges associated with developing gene augmentation therapy for CEP290-associated LCA. In contrast, antisense oligonucleotide therapy does not have these limitations, as these small AONs easily fit into AAVs, and since the endogenous mRNA is the therapeutic target, the maximum expression levels of the wild-type protein will never exceed that of the endogenous protein.

In this study, we employed patient-derived fibroblast cells as a model system to assess the efficacy of AAV-based AON delivery, since they endogenously express the CEP290 protein and develop cilia under serum starvation conditions. Although these cells are hard to transfect with plasmid DNA, transduction of AAVs was more efficient. When testing a panel of different AAV serotypes, AAV2/2 appeared to show the highest tropism for fibroblasts, although AAV2/5 and AAV2/9 also showed affinity for these cells and both AAV2/5 and AAV2/9 have a higher tropism for photoreceptor cells than AAV2/2 (40, 41). Nevertheless, in order to have the optimal vector for the studies in our fibroblast model, AAV2/2 was selected for the generation of the AON-containing vectors. In our therapeutic construct, effective AON sequences were subcloned into a modified U7snRNA gene, a strategy that has previously been shown to be effective in redirecting splicing of the DMD gene (27). It remains to be determined whether the same construct is also effective in redirecting splicing in the context of a photoreceptor cell, ultimately the target cell for therapeutic intervention, but in fibroblast cells this molecular approach appears to be a very effective one.

Despite the suitability of the fibroblast cells as a preclinical model to assess the therapeutic efficacy of AON-based therapy, ideally one would like to study its potential in the context of a living animal, or at least in the context of a photoreceptor cell. We generated a humanized transgenic knock-in mouse model, where part of the mouse Cep290 genomic DNA was replaced by its orthologous human counterpart, i.e. exon 26, intron 26 (including the LCA-causing mutation) and exon 27, allowing us to assess the therapeutic efficacy of AON therapy in vivo, by delivering AONs either as naked molecules or in AAVs. Unfortunately however, despite a correct genetic engineering of the transgenic mouse model, hardly any aberrant splicing of Cep290 mRNA nor a concomitant retinal phenotype was observed in these mice (28), rendering this model inappropriate for further studies. An alternative model that can be used to assess the potential of AON therapy is the induced pluripotent stem cell (iPSC)-derived photoreceptor cells. The discovery that four basic transcription factors can transform fully differentiated adult cells into cells with pluripotent capacity (42) has revolutionized the field of stem cell biology, and has resulted in establishing procedures to successfully differentiate such cells to human photoreceptor-like cells in a culture dish (43-48). Starting with for instance a fibroblast cell line of an IRD patient, it is possible to generate photoreceptor-like cells in the presence of the primary IRD-causing mutation(s), allowing to study the pathophysiological mechanisms that underlie the phenotype (43, 45), as well to assess the efficacy of potential therapeutic strategies in a relevant molecular environment. In the case of CEP290-associated LCA, AONs could be administered to these cells either as naked molecules or packaged in AAVs, and the potential rescue of aberrant CEP290 splicing, CEP290 protein levels, and a ciliary defect could be readily assessed. In addition, delivering AONs to iPSC-derived photoreceptor-like cells provides an opportunity to assess potential off-target effects of the AON via transcriptome sequencing, as these cells show a similar transcriptional profile compared to the real photoreceptors in the human eye. Of note, AONs hybridize to pre-mRNA, are selected to have unique targets and usually a single mismatch already prevents the binding to other targets and hence the ability to interfere with other pre-mRNA splicing events (15).

So far, AONs have been used in clinical trials for other ocular diseases such as CMV-induced retinitis to decrease the viral load in AIDS patients (Vitravene®), or diabetic macula edema and diabetic retinopathy to downregulate c-Raf expression and thereby decrease neovascularization (iCo-007) (21). In addition, a systemic delivery of AONs was recently shown to be successful in a humanized mouse model for Usher syndrome type 1C, characterized by a combination of hearing deficits, vestibular impairment and retinal dystrophy. Intraperitoneal injection of naked AONs targeting a cryptic splice site caused by a recurrent mutation in Ush1C, resulted in an increased level of correctly spliced Ush1C mRNA, and a strong improvement in auditory and vestibular function in these mice (49). A systemic delivery of AONs, whether as naked molecules or packaged in AAVs, however, would require high amounts of AON and increase the chances of evoking immune responses. The fact that the eye is an immune-privileged organ that is tightly regulated to preserve its integrity (50), confers the possibility to deliver the AON molecules directly to the retina, either by intravitreal or subretinal injections with little expected immunological side effects (34).

In terms of future therapeutic intervention in humans, there are several pros and cons to either an AAV-based administration of AONs, or delivery as a naked molecule. Due to their small size, naked AONs may be able to penetrate and reach the photoreceptor cells in the retina more easy upon intravitreal injections, compared to the subretinal delivery of currently used AAVs. Also, as naked AONs have a limited stability (ranging from weeks to months depending on the modifications added to the oligonucleotide), potential negative side effects of the AONs would also disappear after some time. Nevertheless, the use of naked AONs would require multiple, life-long injections, as CEP290-associated LCA manifests already in childhood. In contrast, a single administration of an effective AAV could give therapeutic benefit for many years, potentially life-long. And although with current subretinal surgery, only part of the retina can be targeted, new subclasses of AAVs are currently being developed that would allow a more easy intravitreal delivery, and a more effective targeting of the retina.

One other aspect that determines future therapeutic success, is the preservation of the retina at the time of treatment. Despite the early-onset and severe nature of the visual impairment in CEP290-associated LCA, the integrity of the photoreceptor layer appears to be relatively well conserved in some patients with CEP290-associated LCA, up to young adulthood (51, 52). The same is true for the connections of the visual pathway in the brain, giving hope that these patients are able to process and interpret the visual input that would become available following effective treatment (51). Despite this therapeutic window of opportunity, previous studies have shown a clear correlation between the age of treatment and therapeutic outcome (13), suggesting that LCA patients with CEP290-associated LCA may benefit most from early treatment.

In conclusion, we here show the therapeutic efficacy of AAV-mediated delivery of AONs to treat the most common genetic form of childhood blindness, i.e. CEP290-associated LCA. In fibroblast cells from LCA patients, aberrant CEP290 splicing was corrected, CEP290 protein levels were restored, and a ciliary phenotype was completely rescued following AON administration. This unique combination of splice correction therapy and the use of safe AAV technology, provides an excellent treatment strategy that would require only a single surgical procedure. With that, AON-based therapy could be an effective way to halt the progression or even improve visual function in many severely impaired individuals worldwide.

### REFERENCE LIST EXAMPLE 2

1. R. K. Koenekoop, An overview of Leber congenital amaurosis: a model to understand human retinal development. Survey of ophthalmology 49, 379-398 (2004).
2. E. M. Stone, Leber congenital amaurosis—a model for efficient genetic testing of heterogeneous disorders: LXIV Edward Jackson Memorial Lecture. American journal of ophthalmology 144, 791-811 (2007).
3. A. I. den Hollander, R. Roepman, R. K. Koenekoop, F. P. Cremers, Leber congenital amaurosis: genes, proteins and disease mechanisms. Progress in retinal and eye research 27, 391-419 (2008).
4. F. Coppieters, S. Lefever, B. P. Leroy, E. De Baere, CEP290, a gene with many faces: mutation overview and presentation of CEP290base. Human mutation 31, 1097-1108 (2010).
5. A. I. den Hollander, R. K. Koenekoop, S. Yzer, I. Lopez, M. L. Arends, K. E. Voesenek, M. N. Zonneveld, T. M. Strom, T. Meitinger, H. G. Brunner, C. B. Hoyng, L. I. van den Born, K. Rohrschneider, F. P. Cremers, Mutations in the CEP290 (NPHP6) gene are a frequent cause of Leber congenital amaurosis. American journal of human genetics 79, 556-561 (2006).
6. T. Nagase, K. Ishikawa, D. Nakajima, M. Ohira, N. Seki, N. Miyajima, A. Tanaka, H. Kotani, N. Nomura, O. Ohara, Prediction of the coding sequences of unidentified human genes. VII. The complete sequences of 100 new cDNA clones from brain which can code for large proteins in vitro. DNA research: an international journal for rapid publication of reports on genes and genomes 4, 141-150 (1997).
7. B. Craige, C. C. Tsao, D. R. Diener, Y. Hou, K. F. Lechtreck, J. L. Rosenbaum, G. B. Witman, CEP290 tethers flagellar transition zone microtubules to the membrane and regulates flagellar protein content. The Journal of cell biology 190, 927-940 (2010).
8. F. Coppieters, I. Casteels, F. Meire, S. De Jaegere, S. Hooghe, N. van Regemorter, H. Van Esch, A. Matuleviciene, L. Nunes, V. Meersschaut, S. Walraedt, L. Standaert, P. Coucke, H. Hoeben, H. Y. Kroes, J. Vande Walle, T. de Ravel, B. P. Leroy, E. De Baere, Genetic screening of LCA in Belgium: predominance of CEP290 and identification of potential modifier alleles in AHI1 of CEP290-related phenotypes. Human mutation 31, E1709-1766 (2010).
9. I. Perrault, N. Delphin, S. Hanein, S. Gerber, J. L. Dufier, O. Roche, S. Defoort-Dhellemmes, H. Dollfus, E. Fazzi, A. Munnich, J. Kaplan, J. M. Rozet, Spectrum of NPHP6/CEP290 mutations in Leber congenital amaurosis and delineation of the associated phenotype. Human mutation 28, 416 (2007).
10. W. W. Hauswirth, T. S. Aleman, S. Kaushal, A. V. Cideciyan, S. B. Schwartz, L. Wang, T. J. Conlon, S. L. Boye, T. R. Flotte, B. J. Byrne, S. G. Jacobson, Treatment of leber congenital amaurosis due to RPE65 mutations by ocular subretinal injection of adeno-associated virus gene vector: short-term results of a phase I trial. Human gene therapy 19, 979-990 (2008).
11. S. G. Jacobson, A. V. Cideciyan, R. Ratnakaram, E. Heon, S. B. Schwartz, A. J. Roman, M. C. Peden, T. S. Aleman, S. L. Boye, A. Sumaroka, T. J. Conlon, R. Calcedo, J. J. Pang, K. E. Erger, M. B. Olivares, C. L. Mullins, M. Swider, S. Kaushal, W. J. Feuer, A. Iannaccone, G. A. Fishman, E. M. Stone, B. J. Byrne, W. W. Hauswirth, Gene therapy for leber congenital amaurosis caused by RPE65 mutations: safety and efficacy in 15 children and adults followed up to 3 years. Archives of ophthalmology 130, 9-24 (2012).
12. A. M. Maguire, F. Simonelli, E. A. Pierce, E. N. Pugh, Jr., F. Mingozzi, J. Bennicelli, S. Banfi, K. A. Marshall, F. Testa, E. M. Surace, S. Rossi, A. Lyubarsky, V. R. Arruda, B. Konkle, E. Stone, J. Sun, J. Jacobs, L. Dell'Osso, R. Hertle, J. X. Ma, T. M. Redmond, X. Zhu, B. Hauck, O. Zelenaia, K. S. Shindler, M. G. Maguire, J. F. Wright, N. J. Volpe, J. W. McDonnell, A. Auricchio, K. A. High, J. Bennett, Safety and efficacy of gene transfer for Leber's congenital amaurosis. The New England journal of medicine 358, 2240-2248 (2008).
13. A. M. Maguire, K. A. High, A. Auricchio, J. F. Wright, E. A. Pierce, F. Testa, F. Mingozzi, J. L. Bennicelli, G. S. Ying, S. Rossi, A. Fulton, K. A. Marshall, S. Banfi, D. C. Chung, J. I. Morgan, B. Hauck, O. Zelenaia, X. Zhu, L. Raffini, F. Coppieters, E. De Baere, K. S. Shindler, N. J. Volpe, E. M. Surace, C. Acerra, A. Lyubarsky, T. M. Redmond, E. Stone, J. Sun, J. W. McDonnell, B. P. Leroy, F. Simonelli, J. Bennett, Age-dependent effects of RPE65 gene therapy for Leber's congenital amaurosis: a phase 1 dose-escalation trial. Lancet 374, 1597-1605 (2009).
14. R. E. MacLaren, M. Groppe, A. R. Barnard, C. L. Cottriall, T. Tolmachova, L. Seymour, K. R. Clark, M. J. During, F. P. Cremers, G. C. Black, A. J. Lotery, S. M. Downes, A. R. Webster, M. C. Seabra, Retinal gene therapy in patients with choroideremia: initial findings from a phase 1/2 clinical trial. Lancet 383, 1129-1137 (2014).
15. S. M. Hammond, M. J. Wood, Genetic therapies for RNA mis-splicing diseases. Trends in genetics: TIG 27, 196-205 (2011).
16. J. C. van Deutekom, A. A. Janson, I. B. Ginjaar, W. S. Frankhuizen, A. Aartsma-Rus, M. Bremmer-Bout, J. T. den Dunnen, K. Koop, A. J. van der Kooi, N. M. Goemans, S. J. de Kimpe, P. F. Ekhart, E. H. Venneker, G. J. Platenburg, J. J. Verschuuren, G. J. van Ommen, Local dystrophin restoration with antisense oligonucleotide PRO051. The New England journal of medicine 357, 2677-2686 (2007).
17. N. M. Goemans, M. Tulinius, J. T. van den Akker, B. E. Burm, P. F. Ekhart, N. Heuvelmans, T. Holling, A. A. Janson, G. J. Platenburg, J. A. Sipkens, J. M. Sitsen, A. Aartsma-Rus, G. J. van Ommen, G. Buyse, N. Darin, J. J. Verschuuren, G. V. Campion, S. J. de Kimpe, J. C. van Deutekom, Systemic administration of PRO051 in Duchenne's muscular dystrophy. The New England journal of medicine 364, 1513-1522 (2011).
18. H. P. Erba, H. Sayar, M. Juckett, M. Lahn, V. Andre, S. Callies, S. Schmidt, S. Kadam, J. T. Brandt, D. Van Bockstaele, M. Andreeff, Safety and pharmacokinetics of the antisense oligonucleotide (ASO) LY2181308 as a single-agent or in combination with idarubicin and cytarabine in patients with refractory or relapsed acute myeloid leukemia (AML). Investigational new drugs 31, 1023-1034 (2013).
19. A. W. Tolcher, J. Kuhn, G. Schwartz, A. Patnaik, L. A. Hammond, I. Thompson, H. Fingert, D. Bushnell, S. Malik, J. Kreisberg, E. Izbicka, L. Smetzer, E. K. Rowinsky, A Phase I pharmacokinetic and biological correlative study of oblimersen sodium (genasense, g3139), an antisense oligonucleotide to the bcl-2 mRNA, and of docetaxel in patients with hormone-refractory prostate cancer. Clinical cancer research: an official journal of the American Association for Cancer Research 10, 5048-5057 (2004).
20. C. Gebhard, G. Huard, E. A. Kritikou, J. C. Tardif, Apolipoprotein B antisense inhibition—update on mipomersen. Current pharmaceutical design 19, 3132-3142 (2013).
21. P. Hnik, D. S. Boyer, L. R. Grillone, J. G. Clement, S. P. Henry, E. A. Green, Antisense oligonucleotide therapy in diabetic retinopathy. Journal of diabetes science and technology 3, 924-930 (2009).
22. R. W. Collin, A. I. den Hollander, S. D. van der Velde-Visser, J. Bennicelli, J. Bennett, F. P. Cremers, Antisense Oligonucleotide (AON)-based Therapy for Leber Congenital Amaurosis Caused by a Frequent Mutation in CEP290. Molecular therapy. Nucleic acids 1, e14 (2012).
23. X. Gerard, I. Perrault, S. Hanein, E. Silva, K. Bigot, S. Defoort-Delhemmes, M. Rio, A. Munnich, D. Scherman, J. Kaplan, A. Kichler, J. M. Rozet, AON-mediated Exon Skipping Restores Ciliation in Fibroblasts Harboring the Common Leber Congenital Amaurosis CEP290 Mutation. Molecular therapy. Nucleic acids 1, e29 (2012).
24. N. Dias, C. A. Stein, Antisense oligonucleotides: basic concepts and mechanisms. Molecular cancer therapeutics 1, 347-355 (2002).
25. K. Stieger, J. Schroeder, N. Provost, A. Mendes-Madeira, B. Belbellaa, G. Le Meur, M. Weber, J. Y. Deschamps, B. Lorenz, P. Moullier, F. Rolling, Detection of intact rAAV particles up to 6 years after successful gene transfer in the retina of dogs and primates. Molecular therapy: the journal of the American Society of Gene Therapy 17, 516-523 (2009).
26. G. M. Acland, G. D. Aguirre, J. Bennett, T. S. Aleman, A. V. Cideciyan, J. Bennicelli, N. S. Dejneka, S. E. Pearce-Kelling, A. M. Maguire, K. Palczewski, W. W. Hauswirth, S. G. Jacobson, Long-term restoration of rod and cone vision by single dose rAAV-mediated gene transfer to the retina in a canine model of childhood blindness. Molecular therapy: the journal of the American Society of Gene Therapy 12, 1072-1082 (2005).
27. A. Goyenvalle, A. Vulin, F. Fougerousse, F. Leturcq, J. C. Kaplan, L. Garcia, O.
Dams, Rescue of dystrophic muscle through U7 snRNA-mediated exon skipping. Science 306, 1796-1799 (2004).
28. A. Garanto, S. E. van Beersum, T. A. Peters, R. Roepman, F. P. Cremers, R. W. Collin, Unexpected CEP290 mRNA Splicing in a Humanized Knock-In Mouse Model for Leber Congenital Amaurosis. PloS one 8, e79369 (2013).
29. S. Shafique, S. Siddiqi, M. Schraders, J. Oostrik, H. Ayub, A. Bilal, M. Ajmal, C. Z. Seco, T. M. Strom, A. Mansoor, K. Mazhar, S. T. Shah, A. Hussain, M. Azam, H. Kremer, R. Qamar, Genetic spectrum of autosomal recessive non-syndromic hearing loss in pakistani families. PloS one 9, e100146 (2014).
30. W. S. Rasband, B. U. S. National Institutes of Health, Maryland, USA, Ed. (imagej.nih.gov/ij/, 1997-2012).
31. F. R. Garcia-Gonzalo, K. C. Corbit, M. S. Sirerol-Piquer, G. Ramaswami, E. A. Otto, T. R. Noriega, A. D. Seol, J. F. Robinson, C. L. Bennett, D. J. Josifova, J. M. Garcia-Verdugo, N. Katsanis, F. Hildebrandt, J. F. Reiter, A transition zone complex regulates mammalian ciliogenesis and ciliary membrane composition. Nature genetics 43, 776-784 (2011).
32. O. V. Plotnikova, E. N. Pugacheva, E. A. Golemis, Primary cilia and the cell cycle. Methods in cell biology 94, 137-160 (2009).
33. E. Vallespin, M. A. Lopez-Martinez, D. Cantalapiedra, R. Riveiro-Alvarez, J. Aguirre-Lamban, A. Avila-Fernandez, C. Villaverde, M. J. Trujillo-Tiebas, C. Ayuso, Frequency of CEP290 c.2991_1655A>G mutation in 175 Spanish families affected with Leber congenital amaurosis and early-onset retinitis pigmentosa. Molecular vision 13, 2160-2162 (2007).
34. C. L. Rowe-Rendleman, S. A. Durazo, U. B. Kompella, K. D. Rittenhouse, A. Di Polo, A. L. Weiner, H. E. Grossniklaus, M. I. Naash, A. S. Lewin, A. Horsager, H. F. Edelhauser, Drug and gene delivery to the back of the eye: from bench to bedside. Investigative ophthalmology & visual science 55, 2714-2730 (2014).
35. S. E. Boye, S. L. Boye, A. S. Lewin, W. W. Hauswirth, A comprehensive review of retinal gene therapy. Molecular therapy: the journal of the American Society of Gene Therapy 21, 509-519 (2013).
36. J. Bennett, M. Ashtari, J. Wellman, K. A. Marshall, L. L. Cyckowski, D. C. Chung, S. McCague, E. A. Pierce, Y. Chen, J. L. Bennicelli, X. Zhu, G. S. Ying, J. Sun, J. F. Wright, A. Auricchio, F. Simonelli, K. S. Shindler, F. Mingozzi, K. A. High, A. M. Maguire, AAV2 gene therapy readministration in three adults with congenital blindness. Science translational medicine 4, 120ra115 (2012).
37. P. Colella, I. Trapani, G. Cesi, A. Sommella, A. Manfredi, A. Puppo, C. Iodice, S. Rossi, F. Simonelli, M. Giunti, M. L. Bacci, A. Auricchio, Efficient gene delivery to the cone-enriched pig retina by dual AAV vectors. Gene therapy 21, 450-456 (2014).
38. V. S. Lopes, S. E. Boye, C. M. Louie, S. Boye, F. Dyka, V. Chiodo, H. Fofo, W. W. Hauswirth, D. S. Williams, Retinal gene therapy with a large MYO7A cDNA using adeno-associated virus. Gene therapy 20, 824-833 (2013).
39. E. R. Burnight, L. A. Wiley, A. V. Drack, T. A. Braun, K. R. Anfinson, E. E. Kaalberg, J. A. Halder, L. M. Affatigato, R. F. Mullins, E. M. Stone, B. A. Tucker, CEP290 gene transfer rescues Leber congenital amaurosis cellular phenotype. Gene therapy 21, 662-672 (2014).
40. E. M. Surace, A. Auricchio, Versatility of AAV vectors for retinal gene transfer. Vision research 48, 353-359 (2008).
41. L. H. Vandenberghe, P. Bell, A. M. Maguire, R. Xiao, T. B. Hopkins, R. Grant, J. Bennett, J. M. Wilson, AAV9 targets cone photoreceptors in the nonhuman primate retina. PloS one 8, e53463 (2013).
42. K. Takahashi, S. Yamanaka, Induction of pluripotent stem cells from mouse embryonic and adult fibroblast cultures by defined factors. Cell 126, 663-676 (2006).
43. B. A. Tucker, I. H. Park, S. D. Qi, H. J. Klassen, C. Jiang, J. Yao, S. Redenti, G. Q. Daley, M. J. Young, Transplantation of adult mouse iPS cell-derived photoreceptor precursors restores retinal structure and function in degenerative mice. PloS one 6, e18992 (2011).
44. D. A. Lamba, A. McUsic, R. K. Hirata, P. R. Wang, D. Russell, T. A. Reh, Generation, purification and transplantation of photoreceptors derived from human induced pluripotent stem cells. PloS one 5, e8763 (2010).
45. B. A. Tucker, R. F. Mullins, L. M. Streb, K. Anfinson, M. E. Eyestone, E. Kaalberg, M. J. Riker, A. V. Drack, T. A. Braun, E. M. Stone, Patient-specific iPSC-derived photoreceptor precursor cells as a means to investigate retinitis pigmentosa. eLife 2, e00824 (2013).
46. N. Amirpour, F. Karamali, F. Rabiee, L. Rezaei, E. Esfandiari, S. Razavi, A. Dehghani, H. Razmju, M. H. Nasr-Esfahani, H. Baharvand, Differentiation of human embryonic stem cell-derived retinal progenitors into retinal cells by Sonic hedgehog and/or retinal pigmented epithelium and transplantation into the subretinal space of sodium iodate-injected rabbits. Stem cells and development 21, 42-53 (2012).
47. Y. Seko, N. Azuma, M. Kaneda, K. Nakatani, Y. Miyagawa, Y. Noshiro, R. Kurokawa, H. Okano, A. Umezawa, Derivation of human differential photoreceptor-like cells from the iris by defined combinations of CRX, RX and NEUROD. PloS one 7, e35611 (2012).
48. N. Suzuki, J. Shimizu, K. Takai, N. Arimitsu, Y. Ueda, E. Takada, C. Hirotsu, T. Suzuki, N. Fujiwara, M. Tadokoro, Establishment of retinal progenitor cell clones by transfection with Pax6 gene of mouse induced pluripotent stem (iPS) cells. Neuroscience letters 509, 116-120 (2012).
49. J. J. Lentz, F. M. Jodelka, A. J. Hinrich, K. E. McCaffrey, H. E. Farris, M. J. Spalitta, N. G. Bazan, D. M. Duelli, F. Rigo, M. L. Hastings, Rescue of hearing and vestibular function by antisense oligonucleotides in a mouse model of human deafness. Nature medicine 19, 345-350 (2013).
50. I. Benhar, A. London, M. Schwartz, The privileged immunity of immune privileged organs: the case of the eye. Frontiers in immunology 3, 296 (2012).
51. A. V. Cideciyan, T. S. Aleman, S. G. Jacobson, H. Khanna, A. Sumaroka, G. K. Aguirre, S. B. Schwartz, E. A. Windsor, S. He, B. Chang, E. M. Stone, A. Swaroop, Centrosomal-ciliary gene CEP290/NPHP6 mutations result in blindness with unexpected sparing of photoreceptors and visual brain: implications for therapy of Leber congenital amaurosis. Human mutation 28, 1074-1083 (2007).
52. S. E. Boye, W. C. Huang, A. J. Roman, A. Sumaroka, S. L. Boye, R. C. Ryals, M. B. Olivares, Q. Ruan, B. A. Tucker, E. M. Stone, A. Swaroop, A. V. Cideciyan, W. W. Hauswirth, S. G. Jacobson, Natural history of cone disease in the murine model of Leber congenital amaurosis due to CEP290 mutation: determining the timing and expectation of therapy. PloS one 9, e92928 (2014).

### Example 3: AON's Delivered by Adeno Associated Viral Vectors In Vivo

### Introduction

We aimed to determine the efficacy of AAV-AONs and that of naked AON molecules when delivered into the retina. For that purpose, we used our humanized Cep290 mouse model (Cep290^{lca/lca}). This model contains the human exon 26, intron 26 with the c.2991+1655A>G mutation and exon 27. Previously, we showed that pseudo-exon-X is inserted to only a small proportion of Cep290 transcripts, insufficient to cause a retinal phenotype (1). Fibroblast cells derived from this mouse model were transduced with AAV2/2-AON4 and -AON5, to determine which AAV-AON was most effective in a mouse molecular environment (data not shown). AON5 showed the highest efficacy as was selected for this in vivo study.

### Materials and Methods

### Intraocular Injections

Ten animals were used per molecule (naked molecule (nkdAON), AAV2/9-U7snRNA-NoAON (AAV-NoAON) or AAV2/9-U7snRNA-AON (AAV-AONS). Mice were anesthetized using isofluorene (also during the surgical procedure) and analgesia was injected subcutaneously. Intravitreal (for naked AONs) and subretinal (for AAVs) injections were performed on a humanized Cep290 mouse model carrying the intronic CEp290 mutation [1]. Three microliters of naked AON (20 μg/μl), AAV-NoAON and AAV-AON (˜3×10¹² GC/ml both) were injected in the right eye, whereas 3 microliters of PBS were injected in each left eye. Retinas were harvested ten days post-injection. Eight animals were used for RNA analysis, while two mice were employed to assess retinal morphology. In order to obtain sufficient RNA, two retinas were pooled per group, leading to four biological replicates for each molecule.

### Statistical Analysis

In order to study the differences between treated and untreated cells we applied the two-tailed Student's T and Mann-Whitney tests. P-values smaller than 0.05 were considered significant as indicated in the figures. Statistical analysis was performed for the quantification of the CEP290 protein levels, as well as the ciliation and cilium length measurements. For the assessment of the efficacy in vivo a paired Student's T test was performed to determine statistical significance of the decrease in each replicate, while Student's T and Mann-Whitney tests were used to compare groups.

### Results

AON5 was packaged into an AAV2/9 which presents a high tropism for photoreceptor cells (2, 3). Subsequently, naked AON, AAV2/9-NoAON and AAV2/9-AON5 were delivered to the transgenic mice by intraocular injections. After 10 days, retinas were harvested and analyzed at RNA level. RT-PCR analysis from left eyes (injected with PBS) and right eyes (injected with the naked or AAV-packaged AON) revealed a statistically significant decrease of exon X in the mice treated with naked AONs (p=0.0030), an almost significant decrease for animals injected with AAV-AON5 (p=0.0563) and no differences for the animals that were administered AAV-NoAON (p=0.2413) (FIGS. 9A and 9B). A total of ˜50% and ˜25% reduction of exon-X-containing transcripts was observed for naked AON and AAV-AON5, respectively (FIG. 9C). The amplification of U7snRNA supported the targeting of retinal cells (FIG. 9D). To discard side effects of the delivery of these molecules to the retina, we performed toluidine blue staining. No morphological defects, such as photoreceptor degeneration, were detected (FIG. 10). In addition, to confirm that retinal structure was not compromised, we performed GFAP immunostaining. GFAP expression is an indicator of gliosis and it is considered as a stress marker in the retina (4). No differences were observed between PBS- or molecule-injected eyes (FIG. 11).

### Discussion

Despite the suitability of the fibroblast cells as a preclinical model to assess the therapeutic efficacy of AON-based therapy, ideally one would like to study its potential in the context of a living animal, or at least in the context of a photoreceptor cell. Previously, we generated a humanized transgenic knock-in mouse model where part of the mouse Cep290 genomic DNA was replaced by its orthologous human counterpart, i.e. exon 26, intron 26 (including the LCA-causing mutation) and exon 27, in order to assess the therapeutic efficacy of AON therapy in vivo. However, despite correct genetic engineering of the transgenic mouse model, only a low amount of aberrant splicing of Cep290 mRNA was observed, insufficient to compromise retinal function (1). Yet, the presence of exon-X-containing transcripts did allow to study whether AON administration to the retina of these mice could redirect CEP290 splicing. The delivery of naked AONs was performed via intravitreal injections, since AONs are small molecules that can penetrate all retinal cell layers and thus reach the photoreceptor cells. In contrast, the delivery of AAVs was performed by subretinal injections, with which only a part of the retina is targeted, which increases the variability of each experiment and decreases the apparent efficacy. Indeed, we observed more variability in the decrease of exon-X-containing transcripts in mice treated with AAV-AONs compared to the naked AON molecule, where all replicates showed similar results. In addition, U7 expression levels also supported the variability observed in the delivery of AAVs into murine retina. Yet, although a decrease of only 25-30% of pseudo-exon X may seem not promising, it could well be enough to delay the progression of the disease in humans, since LCA patients carrying this mutation already present 50% of correct transcript, so an increase to ˜70% could be sufficient to halt the photoreceptor degeneration. For instance, in the Cep290^{lca/lca} mouse model, even though 15% of Cep290 transcripts is aberrantly spliced, no morphological or functional problems have been detected (1). Further to this, it should be noted that following subretinal AAV injections, not all photoreceptor cells are targeted and exposed to the AAV, the therapeutic effect may be ‘masked’ by the non-targeted retinal cells that are included in the RT-PCR analysis. Increasing the targeting efficacy (by different surgical procedures) would likely yield a higher transduction efficacy and result in a statistically significant decrease of exon-X-containing transcripts as observed with the (intravitreal) administration of naked AON molecules.

### REFERENCE LIST EXAMPLE 3

1. Garanto, A., et al., Unexpected CEP290 mRNA Splicing in a Humanized Knock-In Mouse Model for Leber Congenital Amaurosis. PLoS One, 2013. 8(11): p. e79369.
2. Vandenberghe, L. H., et al., AAV9 targets cone photoreceptors in the nonhuman primate retina. PLoS One, 2013. 8(1): p. e53463.
3. Watanabe, S., et al., Tropisms of AAV for subretinal delivery to the neonatal mouse retina and its application for in vivo rescue of developmental photoreceptor disorders. PLoS One, 2013. 8(1): p. e54146.
4. Calandrella, N., et al., Carnitine reduces the hpoperoxidative damage of the membrane and apoptosis after induction of cell stress in experimental glaucoma. Cell Death Dis, 2010. 1: p. e62.

1. An exon skipping molecule that binds to and/or is complementary to a polynucleotide with the nucleotide sequence as shown in SEQ ID NO: 6, or a part thereof.

2.-16. (canceled)

17. The exon skipping molecule according to claim 1 that binds to and/or is complementary to SEQ ID NO: 17, SEQ ID NO: 8, SEQ ID NO: 7, or a part of the foregoing sequences.

18. The exon skipping molecule according to claim 1, wherein said molecule is a nucleic acid molecule.

19. An antisense oligonucleotide that is able to induce the skipping of an aberrant 128 nucleotide exon from human CEP290 pre-mRNA, wherein said antisense oligonucleotide is complementary to a polynucleotide with the nucleotide sequence as shown in SEQ ID NO: 17.

20. The exon skipping molecule according to claim 18, wherein said nucleic acid molecule comprises an antisense oligonucleotide.

21. The antisense oligonucleotide according to claim 19, wherein said antisense oligonucleotide has a length from about 8 to about 128 nucleotides.

22. The antisense oligonucleotide according to claim 19, wherein said oligonucleotide comprises or consists of a sequence selected from the group consisting of: SEQ ID NO: 20, SEQ ID NO: 21, SEQ ID NO: 23 and SEQ ID NO: 24, or said oligonucleotide consists of SEQ ID NO: 22.

23. The exon skipping molecule according to claim 18, wherein said nucleic acid molecule comprises an antisense oligonucleotide.

24. The antisense oligonucleotide according to claim 22, wherein a nucleotide in the antisense oligonucleotide may be an RNA residue, a DNA residue, or a nucleotide analogue or equivalent.

25. The exon skipping molecule according to claim 1 comprising a 2′-O alkyl phosphorothioate antisense oligonucleotide, such as 2′-O-methyl modified ribose (RNA), 2′-O ethyl modified ribose, 2′-O-propyl modified ribose, and/or substituted derivatives of these modifications.

26. The antisense oligonucleotide according to claim 19 comprising a 2′-O alkyl phosphorothioate antisense oligonucleotide, such as 2′-O-methyl modified ribose (RNA), 2′-O ethyl modified ribose, 2′-O-propyl modified ribose, and/or substituted derivatives of these modifications.

27. A viral vector expressing the antisense oligonucleotide according to claim 19 when placed under conditions conducive to expression of the molecule.

28. The viral vector according to claim 27, wherein the viral vector is an AAV vector.

29. The viral vector according to claim 28, wherein the AAV vector is an AAV2/5, AAV2/8, AAV2/9 or AAV2/2 vector.

30. A viral vector expressing an exon skipping molecule as defined in claim 1 when placed under conditions conducive to expression of the molecule.

31. The viral vector according to claim 30, wherein the viral vector is an AAV vector.

32. The viral vector according to claim 31, wherein the AAV vector is an AAV2/5, AAV2/8, AAV2/9 or AAV2/2 vector.

33. A pharmaceutical composition comprising an antisense oligonucleotide according to claim 19 and a pharmaceutically acceptable excipient.

34. A pharmaceutical composition comprising a viral vector according to claim 27 and a pharmaceutically acceptable excipient.

35. A method for modulating splicing of CEP290 in a cell, said method comprising contacting said cell with the antisense oligonucleotide according to claim 19.

36. A method for the treatment of a CEP290 related disease or condition requiring modulating splicing of CEP290 of an individual in need thereof, said method comprising administering to said individual the antisense oligonucleotide according to claim 19.

37. The method according to claim 36, wherein the CEP290 related disease or condition is Leber congenital amaurosis.

38. The method according to claim 36, wherein said administration is intraocular.

39. The method according to claim 38, wherein the intraocular administration is intravitreal or subretinal.
